(19) United States
(12) Patent Application Publication
(10) Pub. No.: US 2026/0038681 A1
(43) Pub. Date: Feb. 5, 2026

(54) ADAPTIVE CLOSED-LOOP HEMORRHAGIC SHOCK RESUSCITATION CONTROLLER

(71) Applicants: The Government of the United States, as Represented by the Secretary of the Army, Fort Detrick, MD (US); The State Of Israel, Ministry Of Defence, Tel Hashomer (IL)

(72) Inventors: Saul J. Vega, Fort Detrick, MD (US); Eric J. Snider, Fort Sam Houston, TX (US); Sofia I. Hernández Torres, Fort Sam Houston, TX (US); Guy Avital, Tel Aviv-Jaffa (IL)

(73) Assignees: The Government of the United States, as Represented by the Secretary of the Army, Fort Detrick, MD (US); The State Of Israel, Ministry Of Defence, Tel Hashomer (IL)

(21) Appl. No.: 19/353,921

(22) Filed: Oct. 9, 2025

Related U.S. Application Data
(63) Continuation-in-part of application No. 18/628,287, filed on Apr. 5, 2024.
(60) Provisional application No. 63/518,724, filed on Aug. 10, 2023.

(51) Int. Cl. G16H 40/63; G16H 10/40; G16H 10/60; G16H 50/20; G16H 70/20

(52) U.S. Cl. CPC G16H 40/63; G16H 10/40; G16H 10/60; G16H 50/20; G16H 70/20

(57) ABSTRACT

Methods, devices, systems, and computer-readable media are described that provide supervisory control of physiologic closed-loop controllers including: monitoring in real-time physiological state information of a patient and continuously determining from the physiological state information a plurality of casualty states of the patient; and in response to changes in two or more determined casualty states of the patient, simultaneously adaptively controlling and adjusting operation of a plurality of physiological closed-loop controllers (PCLCs) in accordance with a plurality of supervisory rules of a supervisory algorithm for casualty management (SACM) to harmonize operation of the PCLCs.

### CROSS REFERENCE TO RELATED APPLICATIONS

This application is a continuation-in-part of U.S. application Ser. No. 18/628,287, entitled “Adaptive Closed Loop Hemorrhagic Shock Resuscitation Controller,” and filed on Apr. 5, 2024, which is a non-provisional of U.S. Provisional Application No. 63/518,724, entitled “Evaluation of Adaptive Closed Loop Hemorrhagic Shock Resuscitation Controllers,” and filed Aug. 10, 2023. The contents of the above listed applications are expressly incorporated herein by reference in their entirety for any and all non-limiting purposes.

### FIELD OF USE

Aspects of the disclosure relate generally to systems for hemorrhagic shock resuscitation. More particularly, aspects described herein relate to an adaptive and closed loop hemorrhagic shock resuscitation controller device implementing various algorithms to control an adaptive resuscitation controller and other devices, such as an automated tourniquet.

### BACKGROUND

Given the evolving nature of patient care and military conflicts, there is an ever-present need for processes that automate, streamline, and/or otherwise augment the delivery of medical care in both emergency and combat trauma environments. Along those lines, various simple devices for automated patient care have been devised. For example, an autonomous device might be able to take care of providing ventilation to a patient under certain conditions, offloading the responsibility of this task away from a human medical provider (who can then be tasked with a different role, improving patient care overall). Such devices are generally closed-loop devices: they receive some simplistic input data (e.g., whether the patient is breathing on their own) and make a care decision based on that data (e.g., whether to aid in ventilation). This simplicity has various advantages: it keeps the devices easy to use, relatively predictable and reliable, repairable, and cheap.

With that said, managing multiple automated devices simultaneously, all potentially competing with one another, can be difficult, particularly in an environment where human medical providers might also be providing medical care at the same time. As an example of this problem, each of a variety of different automated patient care devices might be capable of executing independently but might not be aware of a patient's overall status. In turn, the myopic perspective of these devices (and the simple fact that they are not aware of the existence of other devices) could cause devices to provide contradictory care. It is often the case in perioperative settings and intensive care units that, when a patient requires multiple interventions simultaneously, a clinician will prioritize one line of treatment at the expense of another-that said, autonomous systems cannot make similar decisions, as they do not have such insight into the overall status of a patient. This can have undesirable consequences for a patient: after all, in extreme situations, two different caregiving devices locked in inadvertent competition for one another might end up harming, rather than helping, the patient.

### SUMMARY

The following presents a simplified summary of various aspects described herein. This summary is not an extensive overview, and is not intended to identify key or critical elements or to delineate the scope of the claims. The following summary merely presents some concepts in a simplified form as an introductory prelude to the more detailed description provided below.

Aspects described herein relate to an adaptive closed-loop hemorrhagic shock resuscitation controller. This process implements a Supervisory Algorithm for Casualty Management (SACM) device that manages decisions and interplay between various automated systems, such as those designed for the management of hemorrhage control (e.g., an automated tourniquet system, such as an automated tightening tourniquet device) and resuscitation (e.g., an adaptive resuscitation controller). This system advantageously allows the SACM device to implement different approaches to prioritizing patient care by, for example, carefully controlling the infusion of fluids into a patient in view of other factors, such as the application of a tourniquet. This system also monitors physiological inputs for both systems and synchronizes the systems in view of an overall patient status, in effect preventing the systems from conflicting and improving patient care overall. The SACM's process is generally performed in two stages: an active intervention stage (whereby devices are activated and are implemented to some form of a baseline—such as activating a tourniquet and causing it to tighten and try to impede bleeding, or activating an adaptive resuscitation controller and causing it to begin to monitor patient vitals and deliver fluids), and then a patient monitoring stage (whereby the SACM device continually monitors the status of various devices and the patient and, as needed, activates and/or otherwise modifies the operating parameters of the devices to maintain patient care). In this way, the SACM may use optimized algorithms to provide fluid infusions at an optimal rate while simultaneously monitoring and controlling different (albeit related) patient care priorities, such as the inhibition of hemorrhage.

More particularly, a computing device, such as the aforementioned SACM device, may be configured to provide adaptive and closed-loop hemorrhagic shock resuscitation using an automated tourniquet and an adaptive resuscitation controller. The computing device may cause activation of the automated tourniquet by causing the automated tourniquet to increase pressure of a component of the automated tourniquet to impede extremity bleeding of a patient. The computing device may then cause causing activation of the adaptive resuscitation controller by determining a desired mean arterial pressure setpoint and causing, based on the desired mean arterial pressure setpoint, the adaptive resuscitation controller to infuse a first quantity of fluids into the patient. In either or both cases, causing activation of either device may comprise deactivating an automatic functioning of the device, in effect ceding control of operations of those devices to the computing device. The computing device may monitor component pressure measurements corresponding to the component of the automated tourniquet and, based on the component pressure measurements, cause modification of one or more operating parameters of the automated tourniquet. The computing device may also monitor blood pressure parameters of the patient and, based on comparing the blood pressure parameters to the desired mean arterial pressure setpoint, cause the adaptive resuscitation controller to infuse a second quantity of fluids into the patient. The monitoring of both the automated tourniquet and the adaptive resuscitation controller (and any reactions thereto, such as modifying the operating parameters of either) may be performed at the same time (e.g., in parallel). Moreover, various actions by the computing device (e.g., tightening the automated tourniquet) may be performed based on a wide variety of data, such as the blood pressure parameters of the patient, rather than just data typically available to the automated tourniquet.

Aspects described herein thereby allow the computing device to react to circumstances where one or more devices are not providing an adequate level of care. For example, as part of causing modification of the one or more operating parameters of the automated tourniquet, the computing device may detect air pressure oscillations in the component and then increase a volume of air in the component. In this way, if (for instance) the automated tourniquet loosens or otherwise stops adequately impeding hemorrhage, the computing device may react and provide necessary patient care. As another example, the computing device may detect, based on the component pressure measurements, hemorrhage associated with a first appendage of the patient, even in circumstances where the automated tourniquet is configured to impede extremity bleeding of a second appendage of the patient. In this circumstance (e.g., where tightening of the tourniquet would not stop the newly-discovered hemorrhage), the computing device may take various steps, such as causing the adaptive resuscitation controller to cease infusion of fluids.

A computing device implementing such controller may infuse a quantity of fluid into the patient based on a desired mean arterial pressure setpoint. This process may entail determining intermediate mean arterial pressure setpoints for the adaptive resuscitation controller. As will be described further below, this approach has numerous advantages, including adapting to patient changes in blood pressure in response to fluid as well as potentially avoiding providing too much fluid to a patient. For example, the computing device may determine, based on the desired mean arterial pressure setpoint, a plurality of different intermediate mean arterial pressure setpoints. In turn, causing the adaptive resuscitation controller to infuse the first quantity of fluids into the patient may be based on a first intermediate mean arterial pressure setpoint of the plurality of different intermediate mean arterial pressure setpoints, and causing the adaptive resuscitation controller to infuse the additional quantity/quantities of fluids into the patient may be based on a an intermediate mean arterial pressure setpoint of the plurality of different intermediate mean arterial pressure setpoints.

The infusion of fluids by the adaptive resuscitation controller may be based, in whole or in part, on a correction factor. The computing device may determine a correction factor based on a difference between a predicted time of an increase in mean arterial pressure and an actual time of an increase in mean arterial pressure indicated by the blood pressure parameters of the patient. In turn, the fluids infused into the patient may be based on this correction factor. This, as with the above approach of using intermediate mean arterial pressure setpoints, can aid the adaptive resuscitation controller in providing an appropriate amount of fluids to a patient.

The infusion of fluids may be based on mathematical calculations and/or assumptions. For example, the computing device may determine a volume-pressure relationship between one or more of a total fluid volume associated with the patient or a volume of the first quantity of fluids and at least one of the blood pressure parameters, then determine, based on the volume-pressure relationship, a volume of the additional quantities of fluids. Additionally and/or alternatively, the volume of fluids provided to a patient may be determined, in whole or in part, based on a Dubick constant of approximately 0.337 ml/mmHg/kg.

Broadly, the goal of the infusion of fluids may be to bring the patient to a baseline value, such as the aforementioned mean arterial pressure point. In turn, the computing device may, based on determining that the blood pressure parameters are within 0.5% of the desired mean arterial pressure setpoint, cause the adaptive resuscitation controller to cease infusion of fluids.

Moreover, in accordance with additional embodiments presented herein, a system for adaptive supervisory control of physiologic closed-loop controllers includes: a controller in communication with and operable to simultaneously control a plurality of physiological closed-loop controllers (PCLCs) of a corresponding plurality of autonomous devices for automated patient care and to receive in real-time a plurality of physiological state information of a patient detected by the plurality of autonomous devices; and a supervisory algorithm for casualty management (SACM) of the controller that monitors the physiological state information of the patient received by the controller, continuously determines a plurality of casualty states of the patient from the received physiological state information of the patient, and adaptively controls operation of the plurality of physiological closed-loop controllers (PCLCs) responsive to the determined casualty states of the patient, the SACM operable to simultaneously control and adjust operation of the PCLCs responsive to changes in two or more casualty states of the patient to harmonize operation of the PCLCs in accordance with a plurality of supervisory rules of the SACM.

Further, in accordance with these additional embodiments presented herein, a method configured to provide supervisory control of physiologic closed-loop controllers includes: monitoring in real-time physiological state information of a patient and continuously determining from the physiological state information a plurality of casualty states of the patient; and in response to changes in two or more determined casualty states of the patient, simultaneously adaptively controlling and adjusting operation of a plurality of physiological closed-loop controllers (PCLCs) in accordance with a plurality of supervisory rules of a supervisory algorithm for casualty management (SACM) to harmonize operation of the PCLCs.

These features, along with many others, are discussed in greater detail below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure is illustrated by way of example and not limited in the accompanying figures in which like reference numerals indicate similar elements and in which:

FIG. 1 depicts an illustrative system comprising a supervisory algorithm for casualty management device, an adaptive resuscitation controller device, and an automated tightening tourniquet device.

FIG. 2 depicts an illustrative two-stage process for adaptive closed-loop hemorrhagic shock resuscitation, including an active intervention stage and a patient monitoring stage.

FIG. 3 is a flow chart depicting steps for adaptive closed-loop hemorrhagic shock resuscitation using an automated tourniquet and an adaptive resuscitation controller.

FIG. 4 depicts an illustrative adaptive resuscitation controller device implemented in interacting with a physiology simulator of cardiovascular function.

FIG. 5 depicts an illustrative PhysioVessel test platform.

FIG. 6 depicts an illustrative Hardware-In-Loop Testbed for Resuscitation Controller platform for testing an adaptive resuscitation controller for hemorrhagic shock.

FIG. 7A shows a chart showing mean pressure and flow rate over time for a scenario managed by an adaptive resuscitation controller where mean arterial pressure begins at 45 mmHg with an aggressive hemorrhage rate that initially slows and later accelerates.

FIG. 7B shows a chart showing mean pressure and flow rate over time for a scenario managed by an adaptive resuscitation controller where mean arterial pressure begins at 65 mmHg with an aggressive hemorrhage rate that slows.

FIG. 7C shows a chart comparing scores for different adaptive resuscitation controllers.

FIG. 7D depicts a chart showing mean arterial pressure over time for a study for whole blood resuscitation and a two-hour hold in swine, as managed by an adaptive resuscitation controller.

FIG. 8A comprises charts showing whole blood infusion rates comparing baseline and with a 0.5× correction factor.

FIG. 8B comprises charts showing crystalloid infusion rates comparing baseline and with whole blood infusion rates at a 2× correction factor.

FIG. 8C comprises charts showing infusion rates for a whole blood PhysioVessel with a 240 mL/min hemorrhage and a 120 mL/min hemorrhage.

FIG. 8D comprises charts showing infusion rates for a crystalloid Physio Vessel with a 240 mL/min hemorrhage and a 360 mL/min hemorrhage.

FIG. 9A depicts charts showing distal mean arterial pressure, tourniquet pressure, central mean arterial pressure, infusion rate, and fluid balance over time for a first testing scenario.

FIG. 9B depicts charts showing distal mean arterial pressure, tourniquet pressure, central mean arterial pressure, infusion rate, and fluid balance over time for a second testing scenario.

FIG. 9C depicts charts showing distal mean arterial pressure, tourniquet pressure, central mean arterial pressure, infusion rate, and fluid balance over time for a third testing scenario.

FIG. 9D depicts charts showing distal mean arterial pressure, tourniquet pressure, central mean arterial pressure, infusion rate, and fluid balance over time for a fourth testing scenario.

FIG. 9E depicts charts showing distal mean arterial pressure, tourniquet pressure, central mean arterial pressure, infusion rate, and fluid balance over time for a fifth testing scenario.

FIG. 10A comprises charts showing various comparisons of resuscitation paces for different types of controllers.

FIG. 10B shows charts indicating mean pressure and flow rate over time, as well as comparisons of various controllers with respect to target pressure, for a first scenario.

FIG. 10C shows charts indicating mean pressure and flow rate over time, as well as comparisons of various controllers with respect to target pressure, for a second scenario.

FIG. 10D shows charts indicating mean pressure and flow rate over time, as well as comparisons of various controllers with respect to target pressure, for a third scenario.

FIG. 10E shows charts indicating mean pressure and flow rate over time, as well as comparisons of various controllers with respect to target pressure, for a fourth scenario.

FIG. 10F comprises charts comparing various controllers in terms of overshoot, variable infusion, end-state divergence, and overall score.

FIG. 11 depicts an example of a computing device that may be used in implementing one or more aspects of the disclosure in accordance with one or more illustrative aspects discussed herein.

FIG. 12 illustrates a flow diagram of a supervisory algorithm for casualty management (SACM), casualty state classification and PCLC interaction, in accordance with various embodiments presented herein.

FIG. 13 illustrates SACM development in a flow diagram, in accordance with various embodiments presented herein.

FIG. 14 illustrates an example SACM state diagram with respect to various casualty states, in accordance with various embodiments presented herein.

FIG. 15 illustrates an example SACM state diagram with respect to various casualty states with trigger logic, in accordance with various embodiments presented herein.

FIG. 16 illustrates an example casualty state triage flow that may be employed by a SACM, in accordance with various embodiments presented herein.

FIG. 17 depicts examples of various stable or default casualty state to subsequent, new casualty state progressions, together with potential trigger conditions, in accordance with various embodiments presented herein.

FIG. 18 depicts an example SACM preliminary logic table, in accordance with various embodiments presented herein.

FIG. 19A illustrates hemorrhage severity criteria, in accordance with various embodiments presented herein.

FIG. 19B illustrates example vital scores for mild, moderate and severe hemorrhage casualty states, in accordance with various embodiments presented herein.

FIG. 19C illustrates an example scoring system for hemorrhage shock, in accordance with various embodiments presented herein.

FIG. 20 depicts an example logic table for a Stable casualty state and SACM rules thereof for various PCLC, in accordance with various embodiments presented herein.

FIG. 21 depicts an example logic table for Mild Hemorrhage casualty state and SACM rules thereof for various PCLCs, in accordance with various embodiments presented herein.

FIG. 22 depicts an example logic table for Moderate Hemorrhage casualty state and SACM rules for various PCLCs thereof, in accordance with various embodiments presented herein.

FIG. 23 depicts an example logic table for Severe Hemorrhage casualty state and SACM rules for various PCLCs thereof, in accordance with various embodiments presented herein.

FIG. 24 depicts an example logic table for Mild Sepsis casualty state and SACM rules for various PCLCs thereof, in accordance with various embodiments presented herein.

FIG. 25 depicts an example logic table for Severe Sepsis casualty state and SACM rules for various PCLCs thereof, in accordance with various embodiments presented herein.

FIG. 26 depicts an example logic table for TBI casualty state and SACM rules for various PCLCs thereof, in accordance with various embodiments presented herein.

FIGS. 27 and 28 illustrate example logic tables for pre-needle decompression and post-needle decompression new casualty states and SACM rules for various PCLCs thereof, in accordance with various embodiments presented herein.

FIGS. 29 and 30 illustrate example logic tables for an extremity hemorrhage and an internal (abdominal) hemorrhage casualty states, respectively, and SACM rules for various PCLCs thereof, in accordance with various embodiments presented herein.

FIG. 31 depicts an example logic table for PFC casualty state and SACM rules thereof for various PCLCs in accordance with various embodiments presented herein.

FIG. 32 illustrates a system in which a SACM is in communication with and controls operation of a, in accordance with various embodiments presented herein.

FIG. 33 illustrates a system in which a SACM 3240 is in communication with and controls operation of a number of PCLCs that in turn control operation of corresponding autonomous devices for automated patient care, in accordance with various embodiments presented herein.

FIG. 34 illustrates a system in which a SACM controls via logic circuitry a number of PCLCs and associated autonomous devices directed to oxygen ventilation, mechanical ventilation, anesthesia, and tourniquet functionality, in accordance with various embodiments presented herein.

FIG. 35 illustrates a methodology flow diagram in which the physiological state information of a patient is monitored in real-time to continuously determine and update the casualty state(s) of the patient, in accordance with various embodiments presented herein.

FIG. 36 illustrates a methodology flow diagram in which the physiological state information of a patient is monitored in real-time to continuously determine and update the casualty state(s) of the patient and in which changes in two or more casualty states occur in response to trigger conditions or user input, in accordance with various embodiments presented herein.

### DETAILED DESCRIPTION

In the following description of the various embodiments, reference is made to the accompanying drawings, which form a part hereof, and in which is shown by way of illustration various embodiments in which aspects of the disclosure may be practiced. It is to be understood that other embodiments may be utilized and structural and functional modifications may be made without departing from the scope of the present disclosure. Aspects of the disclosure are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed thereafter and equivalents thereof as well as additional items and equivalents thereof.

As an introduction, autonomous and semi-autonomous medical systems may be able to rapidly execute adjustments in care in response to a patient's needs, thereby possibly providing a significant benefit to patient care. Indeed, those rapid adjustments are particularly critical for patient management in the perioperative and intensive care setting. Such devices include, but not limited to, ventilator management devices, fluid resuscitation devices, pain control and sedation devices, and the like. With that said, such devices often operate independently and in a closed-loop manner, their course of care can contradict. While human caregivers often have the ability to, for example, prioritize a single form of care over others, such closed-loop devices lack this functionality. This is particularly the case over time: while devices might be fairly competent at performing simple tasks (e.g., a ventilator management device might start up and begin providing ventilation reasonably well), over time and with the addition of multiple devices running at different times, the complexity becomes significantly more difficult, and the likelihood of device conflict becomes higher. For example, in some circumstances, a sedation-providing device might still try to sedate a patient when other forms of care (e.g., providing a tourniquet) are significantly more important and somewhat contradictory.

Aspects described herein use a Supervisory Algorithm for Casualty Management (SACM) to manage various devices and ensure a consistent and careful application of care, particularly with respect to the provision of infused fluids to a patient. Specifically, aspects disclosed herein describe a process whereby an Adaptive Resuscitation Controller (ARC) device can be used to infuse fluids for a user while an Automated Tightening Tourniquet (aTKT) device is simultaneously used to control patient hemorrhage. The SACM can use particular algorithms (detailed further below) to carefully control this fluid infusion, thereby optimizing patient care. In this manner, both devices can work quickly and in a complementary manner, rather than in a contradictory manner. Broadly, this process is effectuated through a two-stage process. First, the SACM activates each device in a manner which allows the device to bring the patient to (as best as practicable) a baseline: for example, a tourniquet might tighten to impede hemorrhage, whereas an adaptive resuscitation controller might, based on some algorithm, mathematical relationship, or similar calculation process, infuse fluids to, as best as practical, begin to raise the mean arterial pressure of a patient. Then, the SACM might then monitor measurements and selectively activate and/or modify aspects of either device, in effect working to maintain the baseline. The SACM can continually use the aforementioned algorithm, mathematical relationship, or similar calculation process to provide such care, generally ensuring that a patient is brought to a desired baseline (e.g., a desired mean arterial pressure) in a desirable way (e.g., quickly, without excessive overshoot).

As suggested above, one particularly beneficial aspect of the aspects described above is that the processes described herein can implement a variety of algorithmic approaches for maintaining patient care. Of note, the approach described herein can use at least four different approaches to infusing fluids via the adaptive resuscitation controller: Absolute-ARC (A-ARC), which relates total fluid volume in the system to the pressure (p=m* V+P₀), Relative-ARC (R-ARC), which relates a change in volume in the system to a change in pressure (ΔP=m* ΔV+0), Dubick-o-Matic ARC (D-ARC), which relies on the Dubick constant (that is, approximately 0.337 ml/mmHg/kg), and a Dual Fuzzy Logic (DFL) approach. Such approaches can be taken with respect to a linear regression. These approaches are discussed at length below via test data on a test platform.

Aspects described herein improve the functioning of computers at least because the improvements serve to improve the way in which different devices (e.g., automated tourniquet devices, adaptive resuscitation controller devices, ventilator management devices, fluid resuscitation devices, pain control and sedation devices, and the like) operate, particularly where those devices might provide contradictory care. Such devices are often closed-loop systems with incredibly rudimentary functionalities. While such simplicity has advantages (ease of use, ease of repair, cost), it can also mean that multiple such devices can provide contradictory care for a patient, even when monitored by a human care provider. The aspects described herein remedy these and other issues by creating a system with a supervisory device that, in effect, acts as a proverbial traffic cop for the care provided by various devices. The result of such computer improvements is fundamentally better and more consistent patient care. ARC Device

Discussion will begin with a broad overview of an illustrative system for implementing aspects described herein.

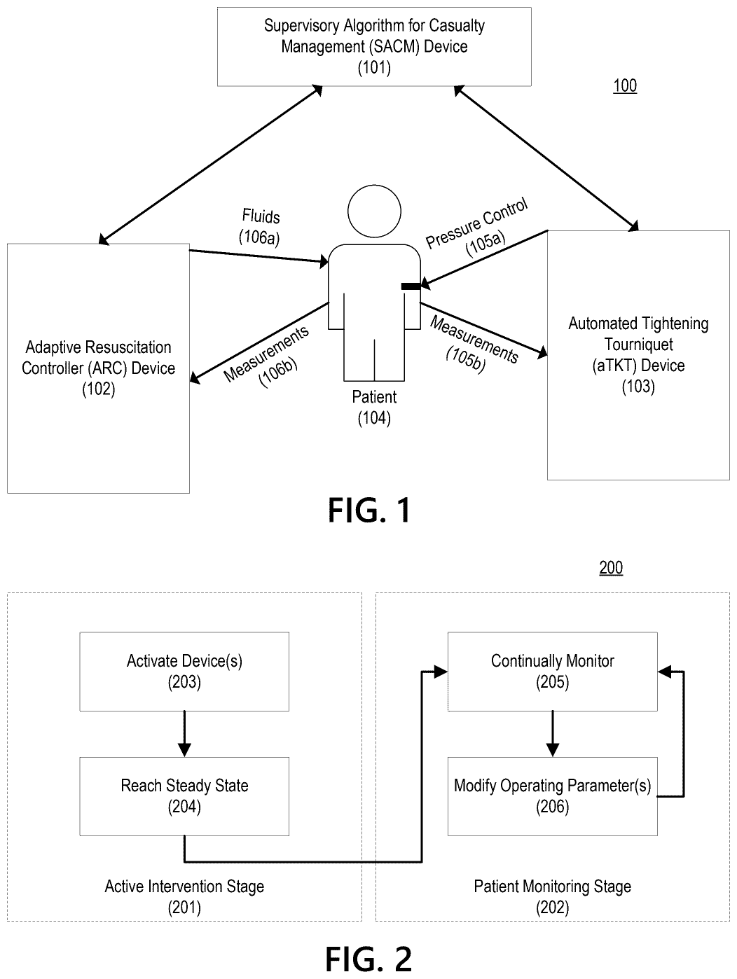

FIG. 1 depicts an illustrative system 100 comprising a Supervisory Algorithm for Casualty Management (SACM) device 101, an Adaptive Resuscitation Controller (ARC) device 102, an Automated Tightening Tourniquet (aTKT) device 103. All such devices are shown with respect to a patient 104. All of these devices may comprise computing devices, such as a computing device comprising one or more processors and memory storing instructions that, when executed by one or more processors, cause various steps to be performed. The ARC device 102 may be configured to deliver fluids to the patient 104 (as represented by arrow 106a) and receive measurements of the patient 104 (as represented by arrow 106b). The aTKT device 103 may be configured to control the pressure of one or more tourniquets (as represented by arrow 105a) and receive measurements of the patient 104 (as represented by arrow 105b).

The SACM device 101 may be configured to manage care of the patient 104 by controlling the functions of the ARC device 102 and the aTKT device 103. Because the patient 104 is being treated by both the ARC device 102 and the aTKT device 103 and are not the same device or communicatively coupled, there may be a possibility that the two devices provide contradictory care to the patient 104. To remedy this issue, the SACM device 101 may be configured to monitor and/or control operations of the ARC device 102 and/or the aTKT device 103 at the same time. As a simple example, the SACM device may be configured to prevent the aTKT device 103 from applying additional pressure to a tourniquet in a manner inconsistent with the provision of fluids by the ARC device 102.

The SACM device 101 may control various devices (such as the ARC device 102 and/or the aTKT device 103) using instructions transmitted to those devices, and the SACM device 101 may receive measurements from those devices using a similar process. For example, one or more of the devices may be connected to one or more networks, such that the devices may communicate (e.g., the SACM device 101 may transmit instructions to the ARC device 102 and/or receive measurements from the aTKT device 103) via the network. With that said, the devices may be communicatively coupled in a variety of ways. For example, the ARC device 102 may connect to the SACM device 101 via a simple Universal Serial Bus (USB) cable or the like.

### Processes

Discussion will now turn to processes which may be performed by devices depicted in FIG. 1, such as the SACM device 101, the ARC device 102, and/or the aTKT device 103.

FIG. 2 depicts an illustrative two-stage process for adaptive closed-loop hemorrhagic shock resuscitation, including an active intervention stage 201 and a patient monitoring stage 202. In the active intervention stage 201, in step 203, a computing device (such as the SACM device 101) may activate one or more devices, such as the ARC device 102 and/or the aTKT device 103. These activated devices may be configured to, upon activation, perform various steps to reach a patient steady state, as reflected in step 204. For example, the ARC device 102 may begin providing fluids, ultimately reaching a desired mean arterial pressure value for the patient. As another example, the aTKT device 103 may be configured to tighten a tourniquet until it has detected that hemorrhaging of a given appendage has been sufficiently impeded or stopped.

In the patient monitoring stage 202, the computing device (e.g., the SACM device 101) may perform various steps to maintain the patient steady state established in step 204. This may include, for example, ensuring that various devices do not fail to operate, that the devices continue to provide any ongoing care necessary for the patient, and the like. In step 205, the computing device may continually monitor measurements taken by the devices (e.g., the measurements taken by the ARC device 102 and/or the aTKT device 103). For example, the computing device may monitor patient blood pressure, the pressure of various tourniquets applied by the aTKT device 103, and the like. Then, in step 206, the computing device may modify operating parameters based on the monitoring performed in step 205. For instance, if the pressure of various tourniquets applied by the aTKT device 103 suggests that a particular tourniquet has become loose, then instructions may be transmitted from the computing device to the aTKT device 103 that cause the aTKT device 103 to increase the tightness of the tourniquet. As another example, if the blood pressure of a patient drops below a threshold, then instructions may be transmitted from the computing device to the ARC device 102 that cause the device to provide additional fluids to the patient.

In both stages, the SACM device 101 may implement an algorithm, mathematical calculation, mathematical constant, or similar processing to control the flow of fluids to a patient. For example, the SACM device 101 may collect data from a variety of data sources (e.g., the ARC device 102, the aTKT device 103, and/or other sources) and make decisions as to how to control the flow rate of an infused fluid (e.g., whole blood, crystalloid fluid) based on that data. In this manner, the SACM device 101 can optimize patient care in a manner that not only avoids device conflict, but also in a manner that potentially expedites bringing a patient to a desired baseline.

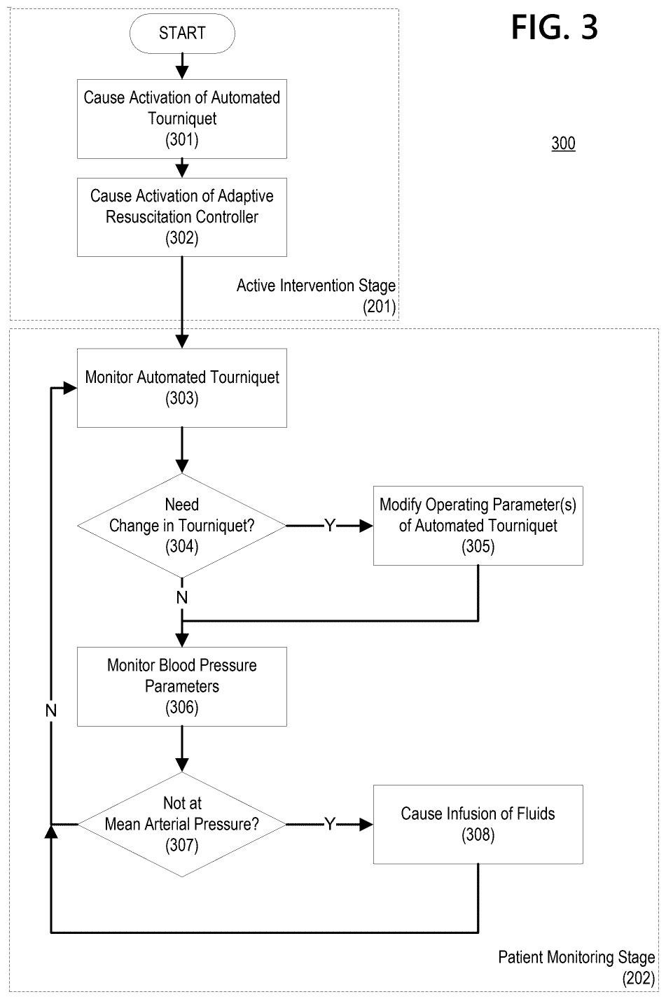

FIG. 3 is a flow chart depicting steps for a method 300 for adaptive closed-loop hemorrhagic shock resuscitation using an automated tourniquet (e.g., the aTKT device 103) and an adaptive resuscitation controller (e.g., the ARC device 102). The steps depicted in FIG. 3 may be performed by one or more computing devices, such as the SACM device 101. A computing device may comprise one or more processors and memory storing instructions that, when executed by the one or more processors, cause performance of one or more of the steps of FIG. 3. One or more non-transitory computer-readable media may store instructions that, when executed, cause a computing device to perform one or more of the steps of FIG. 3. The steps shown in FIG. 3 are illustrative, and might be rearranged or otherwise modified if desired. For instance, various aspects of the later steps of FIG. 3 might be rearranged to account for additional and/or different devices (instead of, as depicted in FIG. 3, an automated tourniquet and/or an adaptive resuscitation controller). Similar to FIG. 2, FIG. 3 begins with the active intervention stage 201 and proceeds to the patient monitoring stage 202.

In step 301, which begins the active intervention stage 201, the computing device may cause activation of an automated tourniquet, such as the aTKT device 103. This step may comprise transmitting instructions to the automated tourniquet and thereby causing it to activate and, as applicable, cause the automated tourniquet to impede extremity bleeding of a patient. For example, the computing device may cause activation of the automated tourniquet by causing the automated tourniquet to increase pressure of a component of the automated tourniquet to impede extremity bleeding of a patient.

In step 302, the computing device may cause activation of an adaptive resuscitation controller, such as the ARC device 102. This process may comprise causing the ARC device 102 to perform steps to bring the patient to a determined desired mean arterial pressure setpoint. For example, the computing device may cause activation of the adaptive resuscitation controller by determining a desired mean arterial pressure setpoint and causing, based on the desired mean arterial pressure setpoint, the adaptive resuscitation controller to infuse a first quantity of fluids into the patient.

As part of determining the desired mean arterial pressure setpoint, the computing device may determine a variety of intermediate mean arterial pressure setpoints. Recognizing that a patient might require an uncertain (and potentially changing) volume of fluids to reach a desired mean arterial pressure, the computing device may determine certain setpoints (e.g., checkpoints) to reach and evaluate how to properly reach (and not undershoot or exceed) the desired mean arterial pressure setpoint. For example, the computing device may determine, based on the desired mean arterial pressure setpoint, a plurality of different intermediate mean arterial pressure setpoints. For instance, for a desired mean arterial pressure setpoint of 65 mmHg and a beginning arterial pressure of 45 mmHg, the computing device may determine different intermediate mean arterial pressure setpoints at increments of 5 mmHg (that is, 50 mmHg, 55 mmHg, 60 mmHg, and finally 65 mmHg). In this manner, the computing device might first provide a first quantity of fluids (e.g., a first bolus of 100 mL over a minute), and then later, based on subsequent measurements, provide a different quantity of fluids (e.g., 90 mL, 110 mL, or the like).

Both step 301 and/or step 302 may involve disabling some or all aspects of the automated tourniquet and/or the adaptive resuscitation controller. Left alone, such devices might operate automatically and without waiting for instructions from the computing device, which might be undesirable in circumstances where the computing device wants to control when (if at all) the devices are operating. As such, the devices might be switched into a manual mode or otherwise configured to activate only upon instruction from the computing device. For example, the computing device may deactivate an automatic functioning of the automated tourniquet and/or may deactivate an automatic functioning of the adaptive resuscitation controller.

After step 302, a patient may have been brought to a relatively safe condition. For example, the automated tourniquet may be (to the extent practicable) impeding hemorrhaging, and the adaptive resuscitation controller may have brought the patient to a desired mean arterial pressure setpoint (or, more practically, might be actively working to bring the patient to that setpoint through the infusion of fluids over some time period).

In step 303, which begins the patient monitoring stage 202, the computing device may monitor the automated tourniquet. This process may comprise monitoring the tightness of (e.g., the pressure of the air inside a bladder of) the tourniquet(s) of the automated tourniquet. For example, the computing device may monitor component pressure measurements corresponding to the component of the automated tourniquet.

Then, in step 304, the computing device may evaluate data collected during the monitoring process of step 303 to determine whether the automated tourniquet should be modified. For instance, a large drop of air (e.g., 33%) might suggest that the tourniquet has become undesirably loose (and/or a mechanical failure), requiring operational changes. Such modification might comprise, for instance, changing operating parameters of the automated tourniquet to tighten, untighten, or otherwise tweak the effectiveness of one or more tourniquets. If there is a needed change in the tourniquet, the method 300 proceeds to step 305. Otherwise, the method 300 proceeds to step 306.

In step 305, and if the computing device determined that a change in the tourniquet was needed, the computing device may modify one or more operating parameters of the automated tourniquet. For example, the computing device may cause, based on the component pressure measurements, modification of one or more operating parameters of the automated tourniquet. As suggested above, this may include tightening, loosening, and/or otherwise modifying one or more components of the tourniquet. Such modification might be further based on blood pressure parameters collected via other devices, such as the ARC device 102. In other words, the tourniquet might be modified based on processing, by the computing device, of a wide variety of data available, not merely the data available via the aTKT device 103.

As an example of step 303, step 304, and step 305, the computing device may detect air pressure oscillations in a component of the automated tourniquet. This may be indicative of a variety of undesirable circumstances, such as the possibility that the tourniquet is not sufficiently tight on an appendage of the patient. In such a circumstance, the computing device might tighten the component by, for instance, increasing a volume of air in the component.

In step 306, the computing device may monitor blood pressure parameters of a patient. Step 306 may involve monitoring a broad variety of aspects of a patient, such as their heart rate, blood pressure, the components of their blood, their heart rate, or the like. Indeed, FIG. 3 focuses on blood pressure merely because it provides a useful example of how the adaptive resuscitation controller might respond to such a change.

In step 307, the computing device may determine whether the blood pressure of the patient (as measured as part of step 306) is at or around the mean arterial pressure. If the blood pressure of the patient is at or around the mean arterial pressure, then the computing device might not need to make a change, and the method 300 might return back to step 303. With that said, if the blood pressure of the patient is not at or around the mean arterial pressure, the method 300 may proceed to step 308.

In step 308, the computing device may cause an infusion of fluids to the patient. For example, the computing device may cause, based on comparing the blood pressure parameters to the desired mean arterial pressure setpoint, the adaptive resuscitation controller to infuse a second quantity of fluids into the patient. The fluids may be infused based on the blood pressure parameters (e.g., as measured via the ARC device 102), the component pressure measurements (e.g., as measured by the aTKT device 103), or the like. In other words, the fluids may be infused based on processing, by the computing device, of a wide variety of data available, not merely the data available via the ARC device 102.

The fluids infused might be based on a correction factor. The correction factor might be based on differences between a predicted time of mean arterial pressure change of a patient and the actual time of an increase in the mean arterial pressure of a patient. Stated differently, the correction factor might account for the fact that the patient's mean arterial pressure might be responding more slowly or more quickly to infused fluids than expected, such that a correction in the quantity of fluids provided to the patient may be necessary. For example, the computing device may determine a correction factor based on a difference between a predicted time of an increase in mean arterial pressure and an actual time of an increase in mean arterial pressure indicated by the blood pressure parameters of the patient, and additional quantities (e.g., a second quantity) of fluids might be based on the correction factor.

Mathematically, the correction factor for a particular time period might be calculated as a function of previous correction factors, a difference in actual and predicted times in which mean arterial pressure was reached, and using a scaling factor (modifiable by an administrator to change the speed of change of a flow rate) as follows:

$$Correction {Factor}_{n + 1} = Correction {Factor}_{n} * \frac{{t}_{actual}}{{t}_{predicted}} * Scaling Factor$$

The fluids infused in both step 302 and 308 might be based on a volume-pressure relationship. The computing device may determine a volume-pressure relationship in at least two ways. On one hand, the volume-pressure relationship may be derived by comparing a total fluid volume associated with a patient with at least one blood pressure parameter. This may yield a total volume-pressure relationship. On the other hand, the volume-pressure relationship may be derived by comparing a volume of a quantity of fluids provided to a patient at a particular time with at least one blood pressure parameter associated with that particular time. This may yield a temporally-based volume-pressure relationship. Either such measurement may be used to determine a quantity of fluids to provide to a patient. In this manner, the computing device might calculate how much fluid to provide to a patient to achieve a desired mean arterial pressure, thereby (to the extent possible) avoiding providing too much or too little fluid at any given time.

Additionally and/or alternatively, the fluids infused in both step 302 and step 308 might be based on a constant, such as an assumed relationship between blood pressure parameters to fluid infusion. For example, the computing device may determine, based on a Dubick constant of approximately 0.337 ml/mmHg/kg, a volume of a quantity of fluids.

In the event that the computing device is providing fluids as part of a plurality of intermediate mean arterial pressure setpoints, the computing device might provide a different quantity of fluids in step 308 as it did in step 302. For example, the computing device might cause the adaptive resuscitation controller to provide a first quantity of fluids based on a first intermediate mean arterial pressure setpoint of the plurality of different intermediate mean arterial pressure setpoints, and then might cause the adaptive resuscitation controller to provide a second quantity of fluids based on a second intermediate mean arterial pressure setpoint of the plurality of different intermediate mean arterial pressure setpoints.

As part of step 308, the computing device may cease infusion of fluids. This may be because a desired mean arterial pressure has been reached. For example, the computing device may, based on determining that the blood pressure parameters are within 0.5% of the desired mean arterial pressure setpoint, cause the adaptive resuscitation controller to cease infusion of fluids. This may additionally and/or alternatively be performed where, for example, data suggests that doing so would be harmful to the patient. For example, the computing device may detect, based on the component pressure measurements, hemorrhage associated with a first appendage of the patient. In such a circumstance, the automated tourniquet may be configured to impede extremity bleeding of a second appendage of the patient. In other words, the automated tourniquet might not be able to, itself, prevent the hemorrhage of the first appendage. In this case, the computing device may cause the adaptive resuscitation controller to cease and/or otherwise modify the infusion of fluids.

As indicated by FIG. 3, step 303 through step 308 may be repeated as desired to continually monitor a patient. For example, the steps depicted in the patient monitoring stage 202 of FIG. 3 may be repeated on a periodic basis (e.g., every ten seconds) so as to ensure that each device monitored by the computing device is providing maximal (and non-contradictory) care to a patient.

Though steps of the patient monitoring stage 202 of FIG. 3 are depicted sequentially, they need not be. For example, steps relating to the automated tourniquet (e.g., step 303, step 304, and/or step 305) may be performed at the same time (e.g., in parallel) with the steps relating to the adaptive resuscitation controller (e.g., step 306, step 307, and/or step 308).

Discussion will now turn to a brief explanation of the ARC device 102.

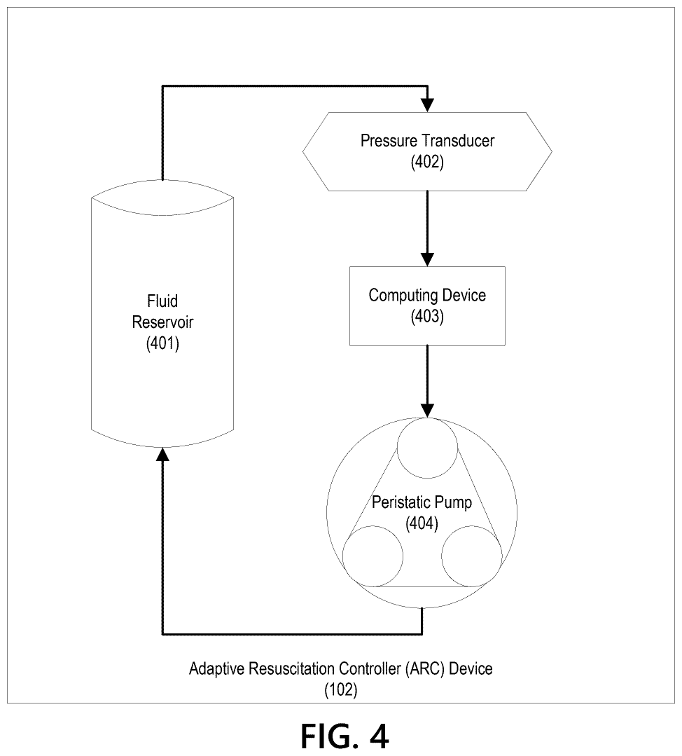

FIG. 4 depicts an illustrative ARC device 102 comprising a fluid reservoir 401, a pressure transducer 402, a computing device 403, and a peristatic pump 404. In the ARC device 102 shown in FIG. 4, a pressure input is sent to the computing device 403 and compared with the desired setpoint, and a flow rate of fluid from the fluid reservoir 401 is adjusted from the setpoint. The computing device 403 is thereby capable of modifying the flow rate by modifying operating parameters of, for example, the pressure transducer 402 and/or the peristatic pump 404.

The ARC device 102 depicted in FIG. 4 may be used to deliver fluids based on instruction(s) received from the SACM device 101. For example, the SACM device 101 may transmit, to the computing device 403 of the ARC device 102, instructions that cause the ARC device 102 to provide a particular quantity of fluids (e.g., 100 mL/min). To implement such instructions, the computing device 403 may control the peristatic pump 404 to pump that volume of liquid from the fluid reservoir 401 and may use the pressure transducer 402 to monitor patient blood pressure and/or the pressure of the fluids exiting the peristatic pump 404.

### Test Environments

Discussion will now turn to various test environments used to develop the above system. The test environments and test data provided below helps underscore, among other things, the unique nature of the innovations described herein, as well as the various ways that the disclosure herein might be implemented (e.g., using different algorithms, different hemorrhage conditions, and the like).

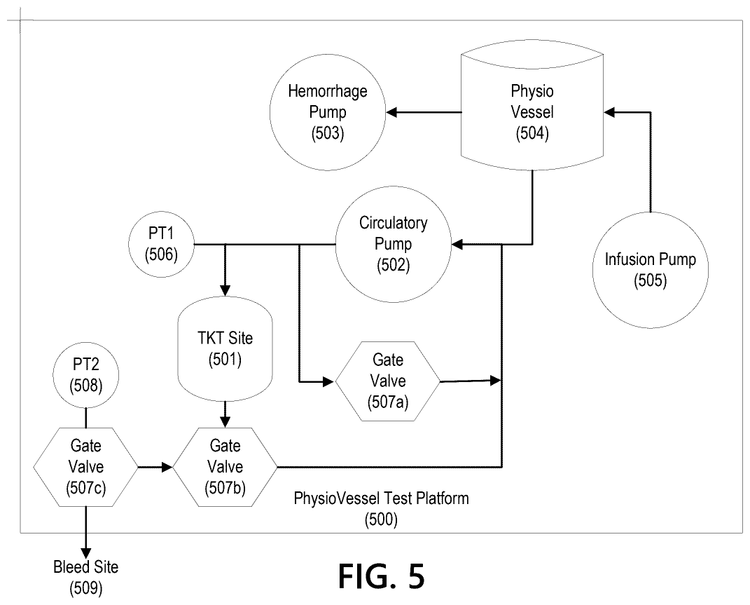

FIG. 5 depicts an illustrative Physio Vessel test platform 500 which can be used to simulate bleeding. In the PhysioVessel test platform 500, various peristaltic pumps (e.g., the circulatory pump 502) and water as the fluid supply may be used to create pulsatile flow through the platform to mimic physiological conditions of a patient. Pressure in the loop may be monitored by a pressure transducer (PT1 506). To enable complete flow occlusion by the tourniquet while permitting continuous flow driven by the circulatory pump 502, a bypass loop may be integrated downstream from PT1 506, with a gate valve 507a allowing tuning to facilitate various circulatory flow rates. A synthetic appendage, such as a phantom arm, might be used to aid in simulation of this system. A t-connector may be used downstream from the synthetic appendage to connect a three-way valve that includes a second pressure transducer (PT2 508) and a bleed site 509. Upstream from the circulatory pump 502 may be a t-connector that integrates a Physio Vessel 504 with a flow loop to provide both volume and pressure to the system. Hydrostatic pressure may be supplied in this system based on the height of the fluid column within the PhysioVessel 504. The relationship between the system volume and this height, and, thus, the pressure within the flow loop, can be precisely controlled to mimic normalized physiological relationships between volume and pressure seen in previous resuscitation studies. Moreover, the hemorrhage pump 503 and the infusion pump 505 may be used to simulate, as desired, internal hemorrhage and infusion of fluid from the adaptive resuscitation controller.

The Physio Vessel test platform 500 may be implemented in at least two ways. First, the PhysioVessel test platform 500 may be implemented as a “whole blood PhysioVessel,” assuming that the infused fluid is whole blood. Second, the Physio Vessel test platform 500 may be implemented as a “crystalloid PhysioVessel,” such that the infused fluid is a crystalloid solution. Various examples of these different Physio Vessels will be described below with respect to different test conditions.

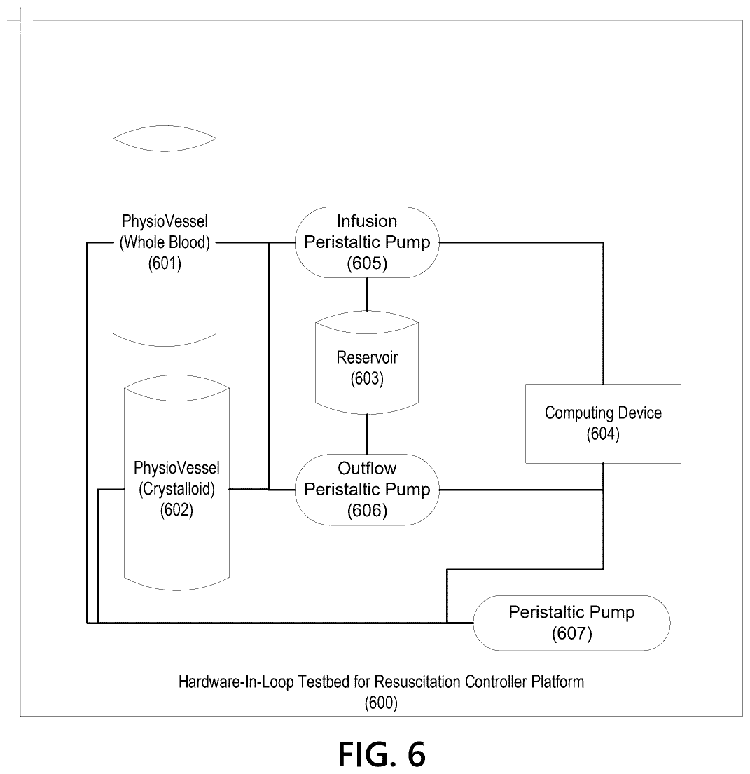

FIG. 6 depicts an illustrative Hardware-In-Loop Testbed for Resuscitation Controller (HATRC) platform 600 depicting two PhysioVessels: a whole blood Physio Vessel 601 and a crystalloid Physio Vessel 602. The whole blood PhysioVessel 601 and the crystalloid PhysioVessel 602 are connected to an infusion peristaltic pump 605 and an outflow peristaltic pump 606, which are both connected to a reservoir 603. The whole blood Physio Vessel 601 and the crystalloid PhysioVessel 602 are also connected to a Peristaltic Pump 607. The infusion peristaltic pump 605, the outflow peristaltic pump 606, and the Peristaltic Pump 607 are all connected to a computing device 604 configured to control the pumps and monitor measurements. The computing device 604 may also be communicatively coupled to various aspects of the Physio Vessels, allowing it to measure, for example, readings from the PT1 506 and/or the PT2 508.

The HATRC platform 600 was the basis for significant testing, including many of the test results provided below. In short, the platform provided a useful tool for emulating human circulatory responses (e.g., blood pressure changes) to, for example, the infusion of fluids, the hemorrhage of those fluids, and the like.

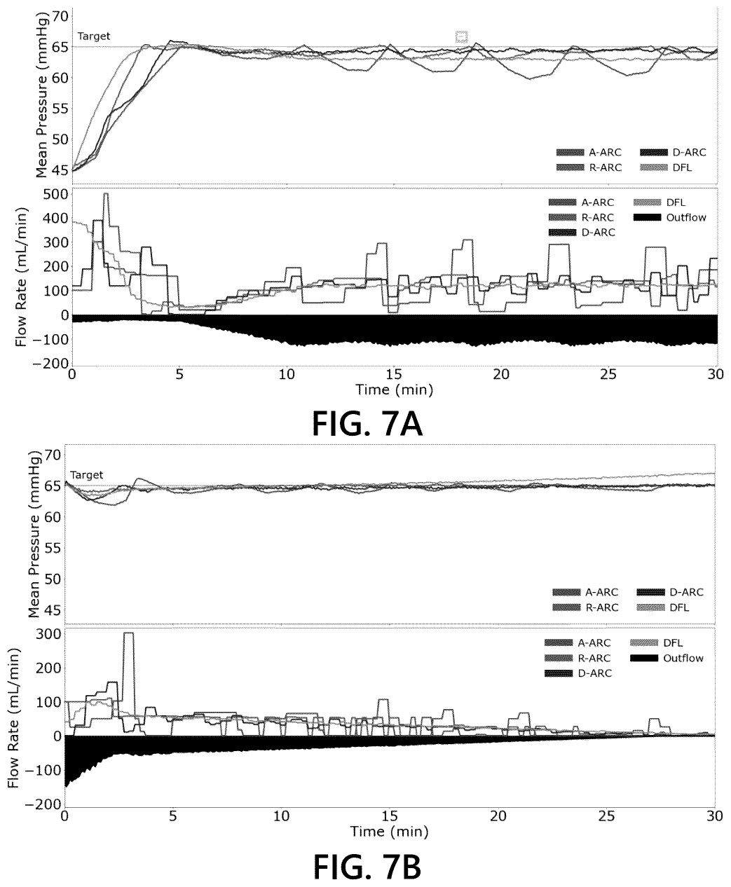

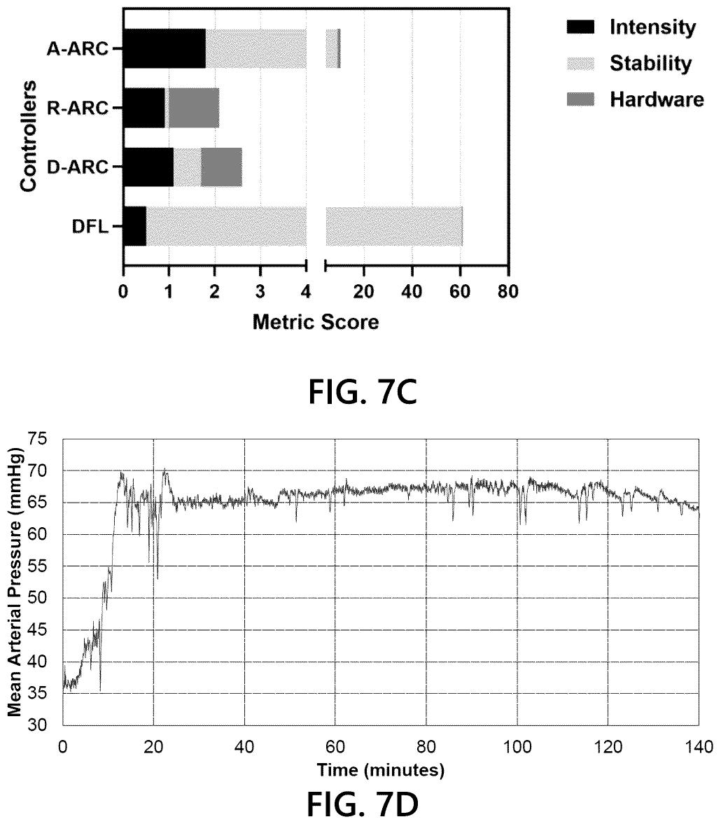

The efficacy of various different algorithms for the ARC device 102 was tested using the aforementioned HATRC platform 600. FIG. 7A shows a chart showing mean pressure and flow rate over time, measured via the HATRC platform 600, for a scenario where mean arterial pressure begins at 45 mmHg with an aggressive hemorrhage rate that initially slows and later accelerates and where the ARC device 102 implements a variety of different algorithms. FIG. 7B is shows a chart showing mean pressure and flow rate over time for the same HATRC platform 600 and for the same algorithms for a scenario where mean arterial pressure begins at 65 mmHg with an aggressive hemorrhage rate that slows. These algorithms include Absolute-ARC (A-ARC), which relates total fluid volume in the system to the pressure (p=m*V+P₀), Relative-ARC (R-ARC), which relates a change in volume in the system to a change in pressure (ΔP=m* ΔV+0), Dubick-o-Matic ARC (D-ARC), which relies on the Dubick constant (that is, approximately 0.337 ml/mmHg/kg), and Dual Fuzzy Logic (DFL) approaches. Such approaches are implemented in this context in a linear regression. Outflow is also shown.

FIG. 7C shows a chart comparing scores for different system controllers (that is, algorithms implemented by the ARC device 102. In this context, lower scores indicate a higher-performing controller, with scores aggregated in terms of intensity, stability, and hardware. This chart generally shows that purpose-built controller designs (e.g., R-ARC, D-ARC) are significantly better than those implementing fuzzy logic, and that in particular the R-ARC algorithm outperformed other designs when tested on the HATRC platform 600.

The efficacy of the R-ARC algorithm as implemented by the ARC device 102 has been shown in a swine study. FIG. 7D depicts a chart showing mean arterial pressure over time for a study for whole blood resuscitation and a two-hour hold in swine using Lactated Ringer's solution. As depicted in this chart, the R-ARC algorithm was able to reach a target MAP (here, 65 mmHg) somewhat quickly, and was able to hold MAP for two hours.

Discussion will now turn to additional testing and results of the ARC device 102, with a particular focus on different PhysioVessels (e.g., the PhysioVessel Test Platform 500, configured to infuse whole blood and/or crystalloid solution).

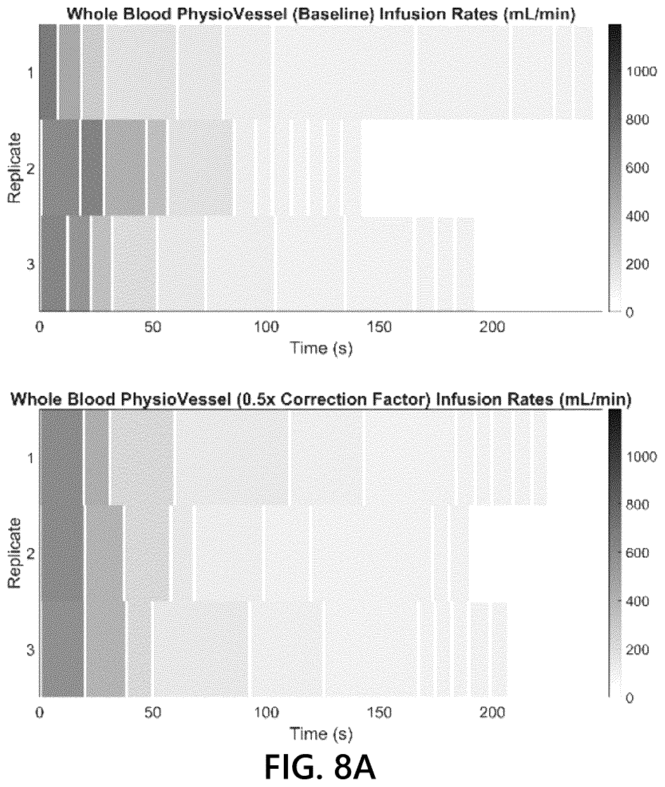

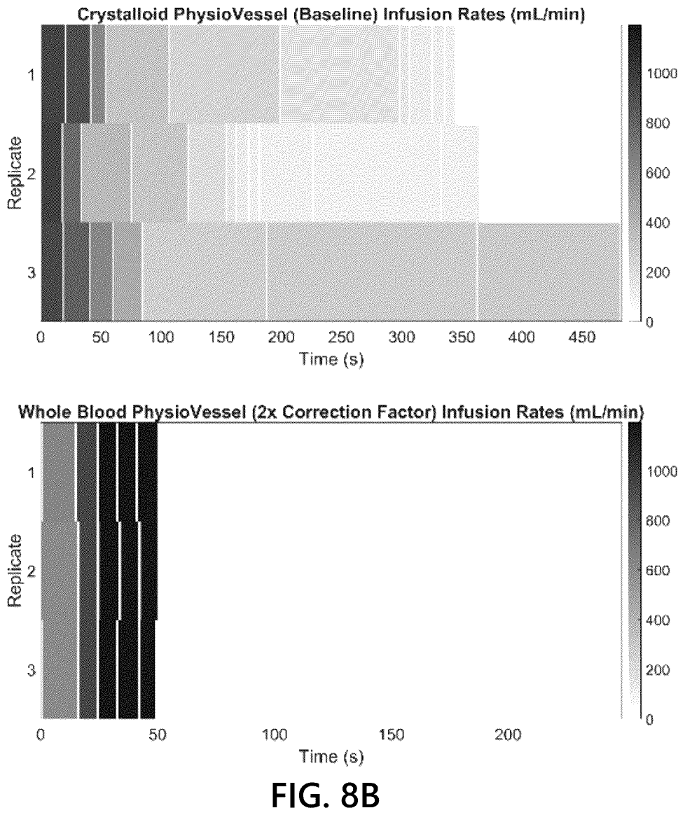

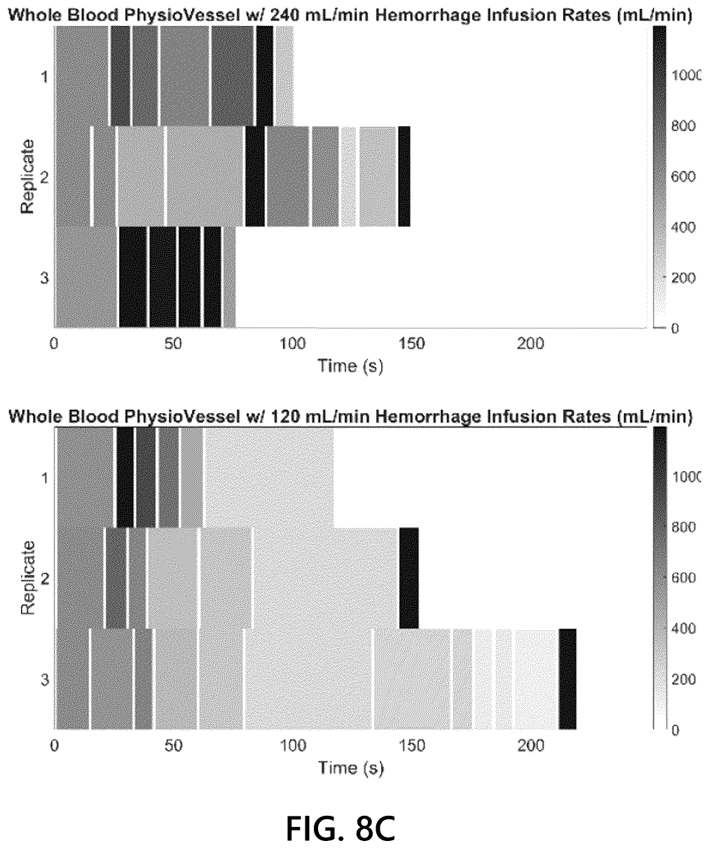

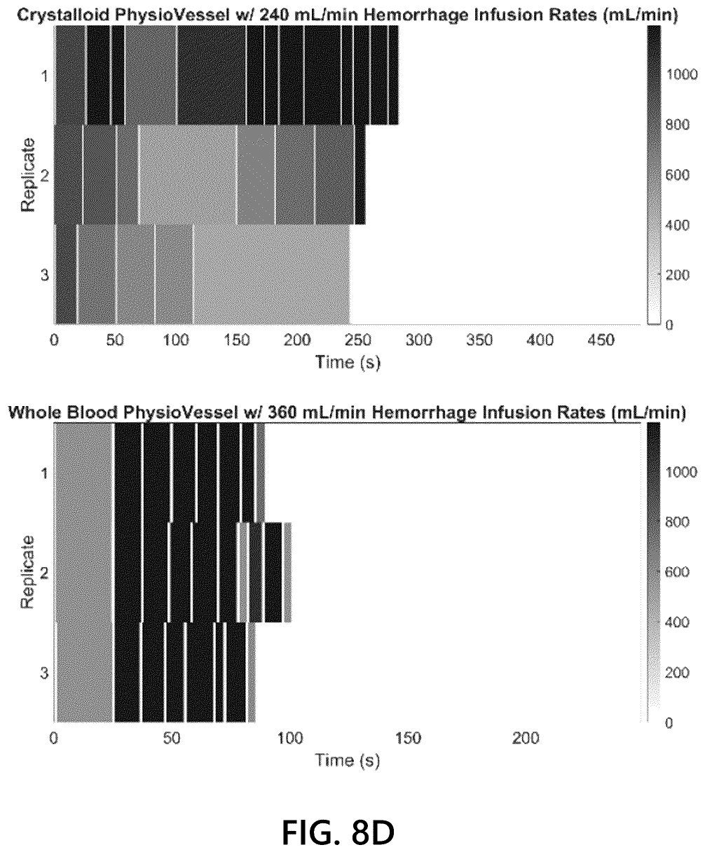

FIG. 8A comprises charts showing whole blood infusion rates comparing baseline and with a 0.5× correction factor. FIG. 8B comprises charts showing crystalloid infusion rates comparing baseline and whole blood infusion rates with a 2× correction factor. These results generally show that, for whole blood, initially high flow rates would reduce to near minimum values as a target setpoint was reached, and that similar results were evident with crystalloid at baseline (although flow rates did not drop as low and required more time to reach the setpoint). When modified, the correction factor changed this quite a bit: for example, when the factor was increased to 2, the flow rate increased and held to the maximum 1200 mL/min almost immediately.

FIG. 8C comprises charts showing infusion rates for a whole blood PhysioVessel with a 240 mL/min hemorrhage and a 120 mL/min hemorrhage. FIG. 8D comprises charts showing infusion rates for a crystalloid PhysioVessel with a 240 mL/min hemorrhage and a 360 mL/min hemorrhage. These results generally show the effect of an internal hemorrhage on the ARC device 102. In these charts, three technical replicates are shown to illustrate how step sizes and flow rate changes varied. Much of the delay in responsiveness between these approaches can be attributed to variability in switching in and out of hemorrhage detection, which was slightly less pronounced for whole blood as compared to crystalloid.

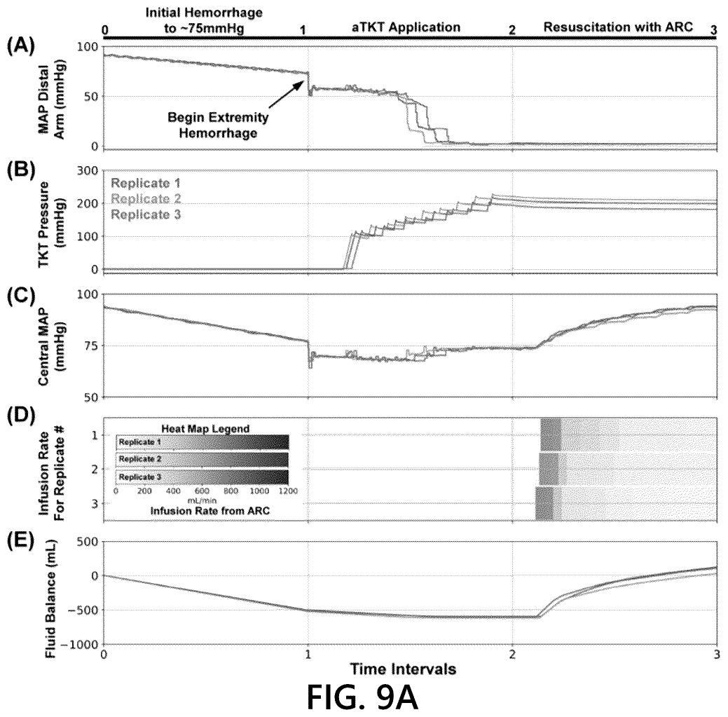

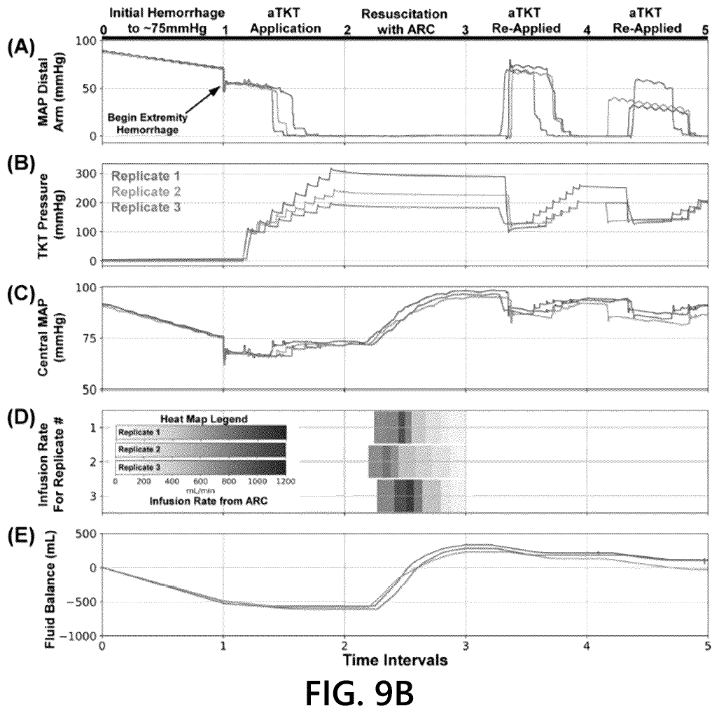

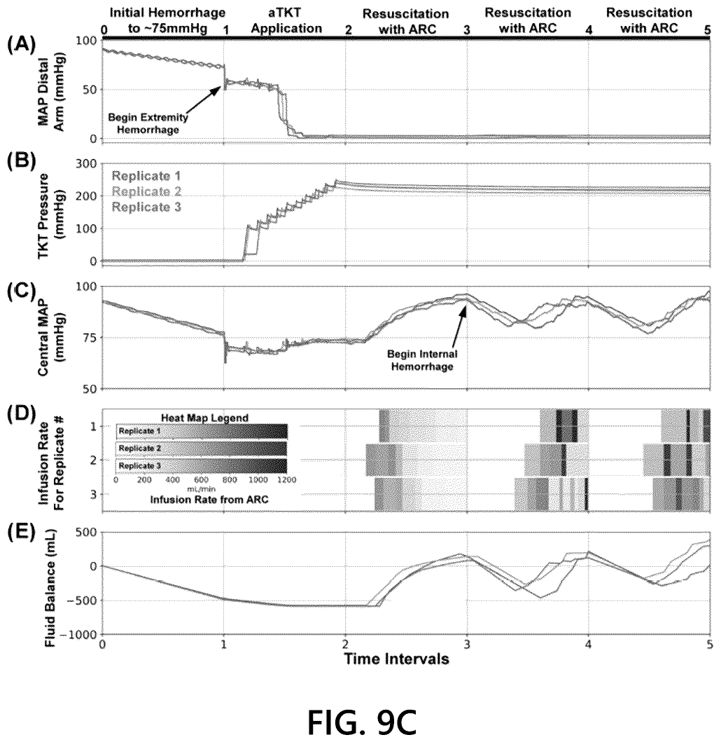

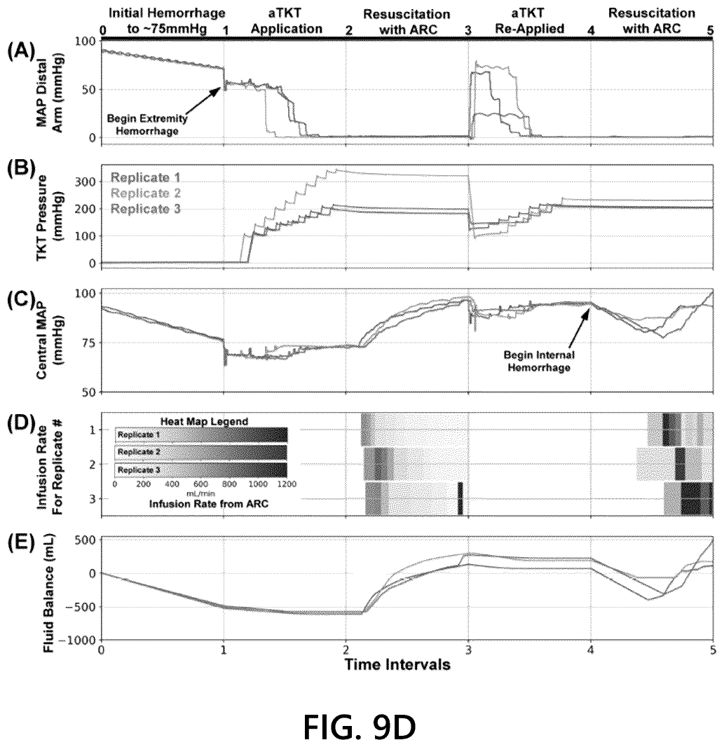

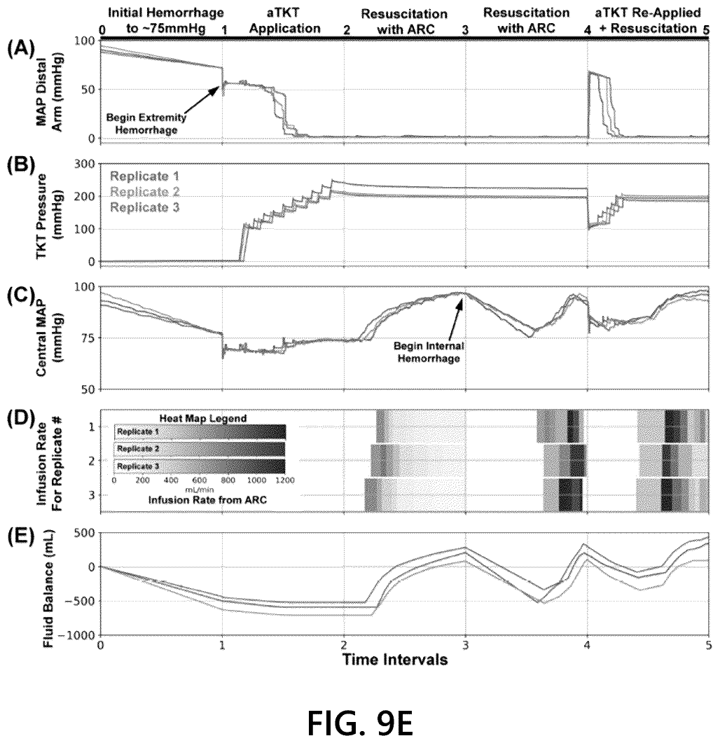

Discussion will now turn to various testing scenarios, illustrating, for different replicates, measurements such as distal mean arterial pressure, tourniquet pressure, central mean arterial pressure, infusion rate, and fluid balance. FIG. 9A depicts charts showing distal mean arterial pressure, tourniquet pressure, central mean arterial pressure, infusion rate, and fluid balance over time for a first testing scenario, wherein an initial hemorrhage (stage 1) was followed by extremity bleed and application of an automated tourniquet (stage 2) and ARC resuscitation (stage 3). FIG. 9B depicts charts showing distal mean arterial pressure, tourniquet pressure, central mean arterial pressure, infusion rate, and fluid balance over time for a second testing scenario, wherein an initial hemorrhage (stage 1) was followed by extremity bleed and application of an automated tourniquet (stage 2), ARC resuscitation (stage 3), and two stages of internal hemorrhage with ARC resuscitation (stages 4-5). FIG. 9C depicts charts showing distal mean arterial pressure, tourniquet pressure, central mean arterial pressure, infusion rate, and fluid balance over time for a third testing scenario, wherein an initial hemorrhage (stage 1) was followed by extremity bleed and application of an automated tourniquet (stage 2), ARC resuscitation (stage 3), the loosening and re-tightening of a tourniquet (stage 4), and (again) the loosening and re-tightening of a tourniquet (stage 5). FIG. 9D depicts charts showing distal mean arterial pressure, tourniquet pressure, central mean arterial pressure, infusion rate, and fluid balance over time for a fourth testing scenario, wherein an initial hemorrhage (stage 1) was followed by extremity bleed and application of an automated tourniquet (stage 2), ARC resuscitation (stage 3), the loosening and re-tightening of a tourniquet (stage 4), and then internal hemorrhage and ARC resuscitation (stage 5). FIG. 9E depicts charts showing distal mean arterial pressure, tourniquet pressure, central mean arterial pressure, infusion rate, and fluid balance over time for a fifth testing scenario, wherein an initial hemorrhage (stage 1) was followed by extremity bleed and application of an automated tourniquet (stage 2), ARC resuscitation (stage 3), internal hemorrhage and ARC resuscitation (stage 4), and then the loosening and re-tightening of a tourniquet (stage 5).

These latter charts show, with particularity, how aspects described herein are uniquely positioned to handle long-term patient management. For examples, the scenarios depicting the loosening and re-tightening of a tourniquet underscore the ability of the SACM device 101 to respond to changes in patient care and respond to those changes, even when other devices (e.g., the ARC device 102) are working.

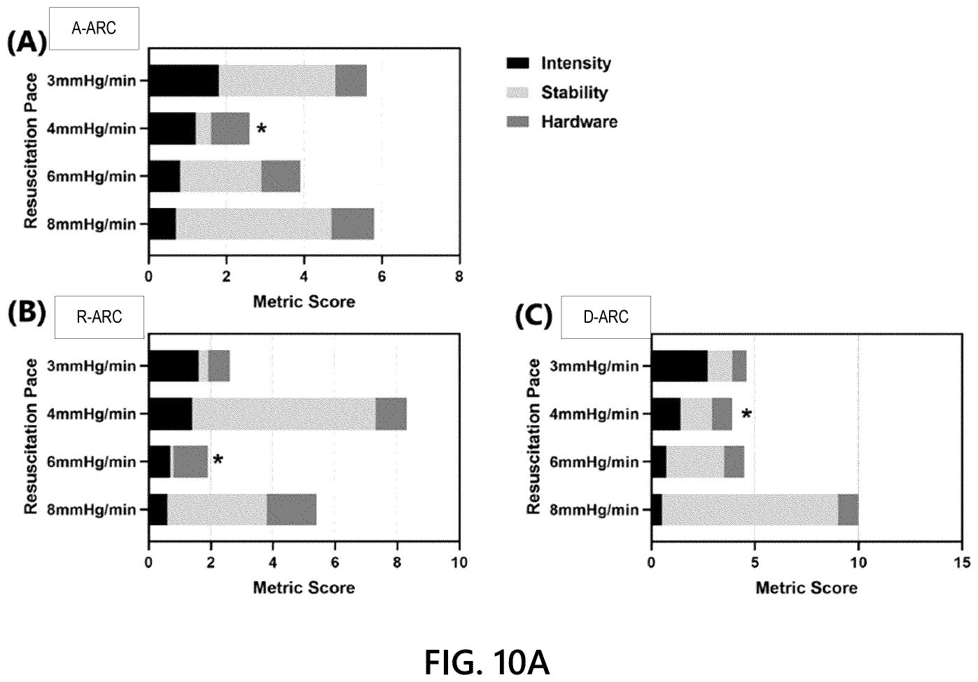

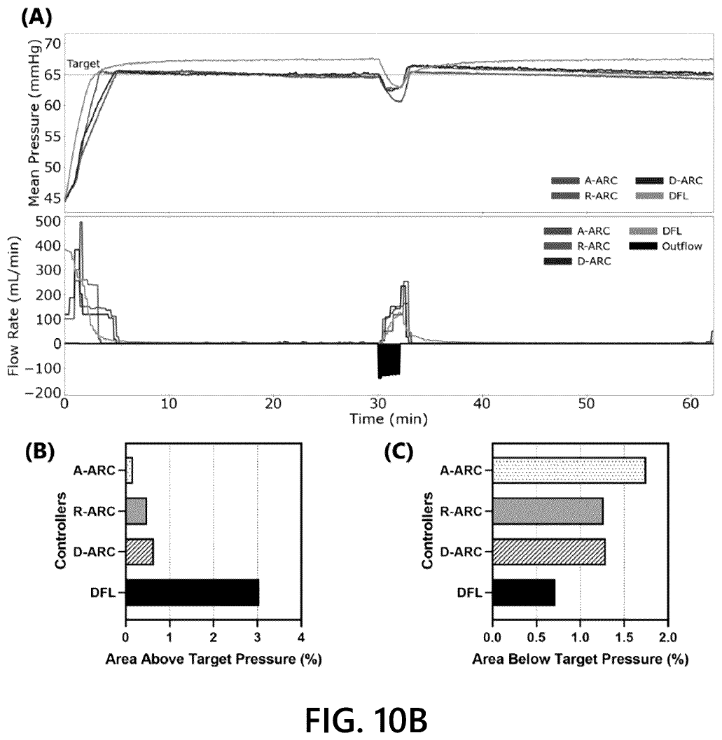

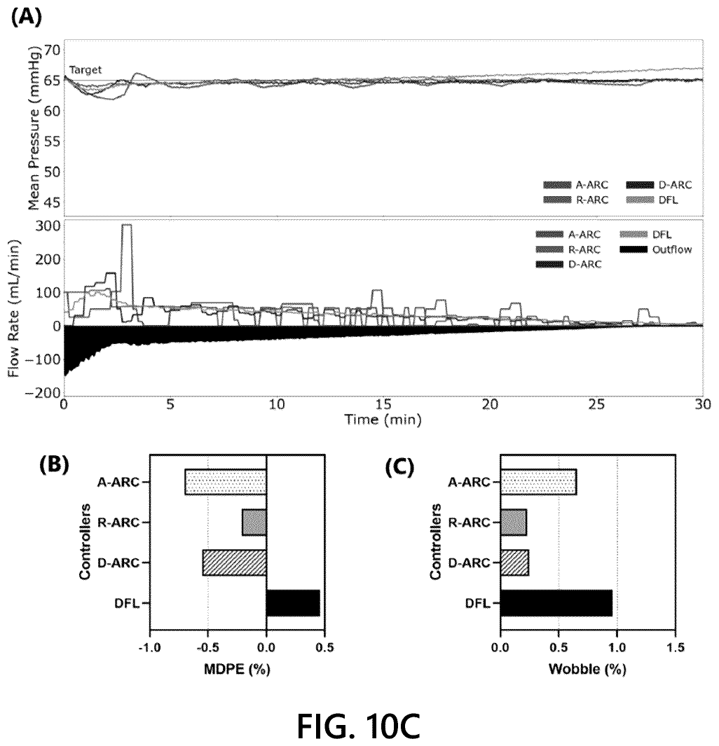

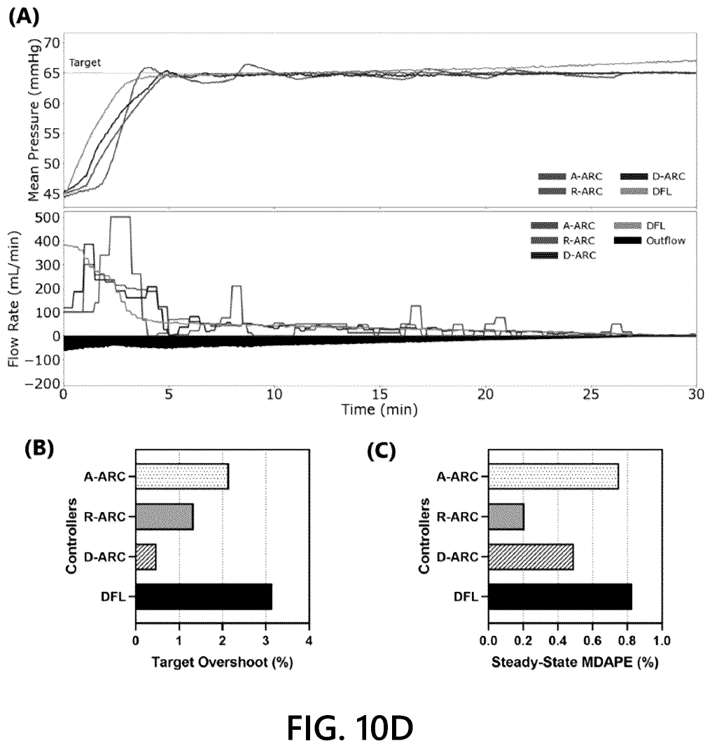

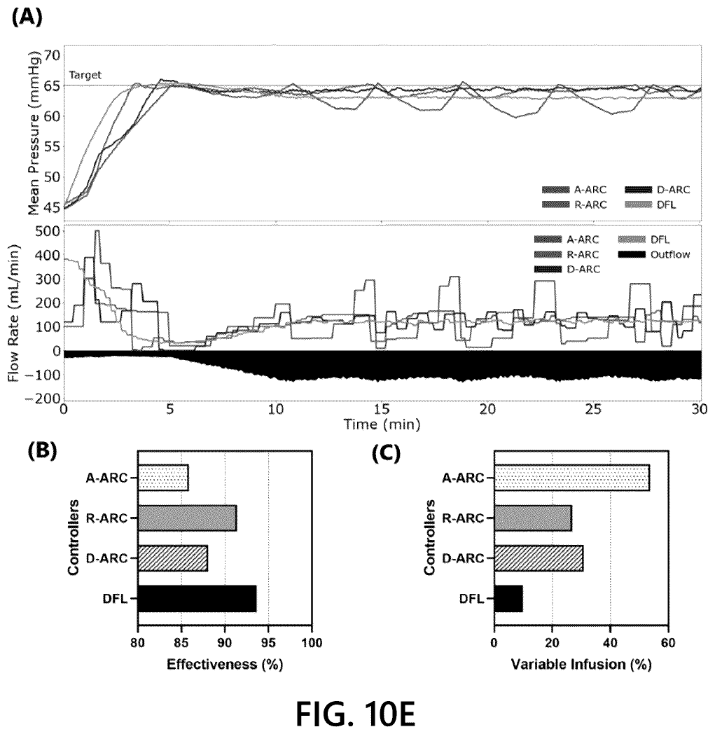

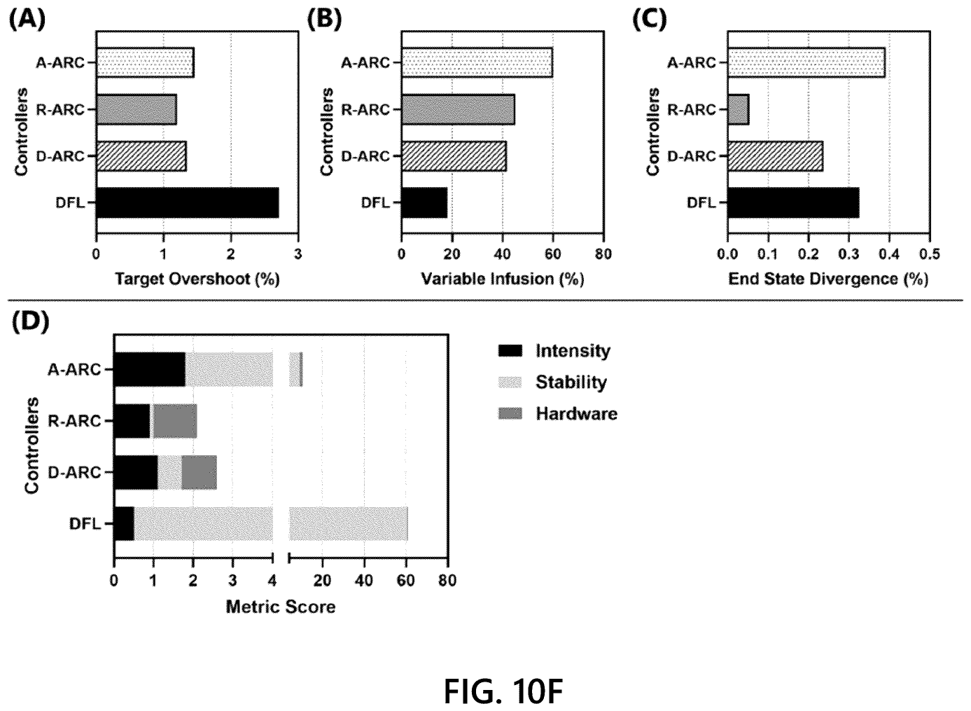

Discussion will now turn to deeper analysis of the various algorithms that may be implemented, by the SACM device 101 and/or the ARC device 102. These include, as described above, Absolute-ARC (A-ARC), which relates total fluid volume in the system to the pressure (p=m*V+P₀), Relative-ARC (R-ARC), which relates a change in volume in the system to a change in pressure (AP=m * ΔV+0), Dubick-o-Matic ARC (D-ARC), which relies on the Dubick constant (that is, approximately 0.337 ml/mmHg/kg), and a Dual Fuzzy Logic (DFL) approach. FIG. 10A comprises charts showing various comparisons of resuscitation paces for the A-ARC algorithm, the R-ARC algorithm, and the D-ARC algorithm, with lower values being better (e.g., faster). This chart shows that, for A-ARC, the 4 mmHg/min version performed best, for R-ARC, the 6 mmHg/min version had the lowest aggregate scores, and for D-ARC, performance was more consistent; however, the 4 mmHg/min had the lowest aggregate metric total.

Each of these algorithms were then tested using four different scenarios. In the first scenario, mean arterial pressure begins at 45 mmHg with no active bleed and a rapid hemorrhage occurs at thirty minutes and lasts for two minutes, with no additional bleed. In the second scenario, mean arterial pressure begins at 65 mmHg with an aggressive hemorrhage rate (maximum˜125 mL/min) that slows throughout the scenario. The third scenario is similar to the second scenario, except that mean arterial pressure begins at 45 mmHg. In the fourth scenario, the mean arterial pressure begins at 45 mmHg but, after five minutes, bleeding accelerates to a maximum rate of ˜125 mL/min and holds at that rate for the remainder of the scenario. FIG. 10B shows charts indicating mean pressure and flow rate over time, as well as comparisons of various controllers with respect to target pressure, for a first scenario (mean arterial pressure begins at 45 mmHg with no active bleed and a rapid hemorrhage occurs at 30 minutes and lasts for 2 minutes, with no additional bleed). FIG. 10C shows charts indicating mean pressure and flow rate over time, as well as comparisons of various controllers with respect to target pressure, for a second scenario (mean arterial pressure begins at 65 mmHg with an aggressive hemorrhage rate (maximum ˜125 mL/min) that slows throughout the scenario). FIG. 10D shows charts indicating mean pressure and flow rate over time, as well as comparisons of various controllers with respect to target pressure, for a third scenario (similar to the second scenario, except that mean arterial pressure begins at 45 mmHg). FIG. 10E shows charts indicating mean pressure and flow rate over time, as well as comparisons of various controllers with respect to target pressure, for a fourth scenario (mean arterial pressure begins at 45 mmHg but, after five minutes, bleeding accelerates to a maximum rate of ˜125 mL/min and holds at that rate for the remainder of the scenario).

FIG. 10F comprises charts comparing various controllers in terms of overshoot, variable infusion, end-state divergence, and overall score. In this case, lower is better. As can be seen from the charts, the R-ARC algorithm generally performed in the most desirable way, having the lowest target overshoot percentage and a significantly lower end state divergence, although it did exhibit a significant degree of variability in infusion.

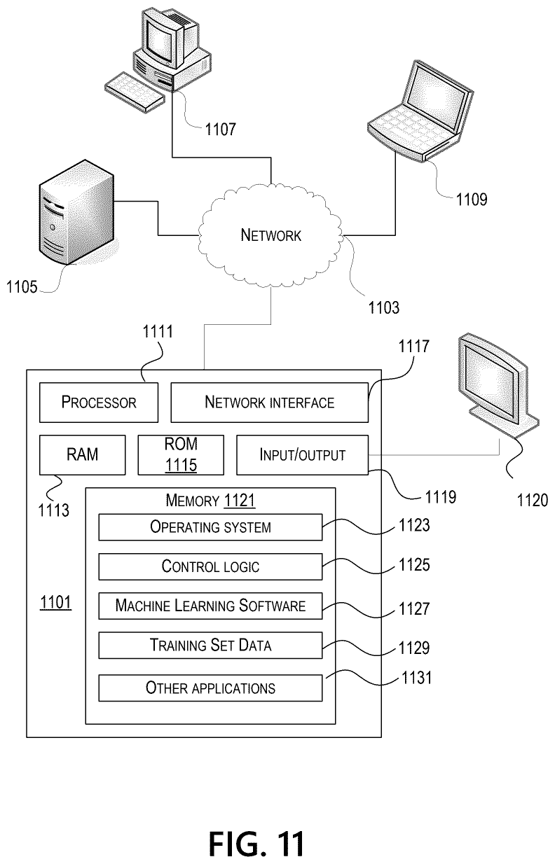

FIG. 11 illustrates one example of a computing device 1101 that may be used to implement one or more illustrative aspects discussed herein. For example, computing device 1101 may, in some embodiments, implement one or more aspects of the disclosure by reading and/or executing instructions and performing one or more actions based on the instructions. In some embodiments, computing device 1101 may represent, be incorporated in, and/or include various devices such as a desktop computer, a computer server, a mobile device (e.g., a laptop computer, a tablet computer, a smart phone, any other types of mobile computing devices, and the like), and/or any other type of data processing device.

Computing device 1101 may, in some embodiments, operate in a standalone environment. In others, computing device 1101 may operate in a networked environment. As shown in FIG. 11, computing devices 1101, 1105, 1107, and 1109 may be interconnected via a network 1103, such as the Internet. Other networks may also or alternatively be used, including private intranets, corporate networks, LANs, wireless networks, personal networks (PAN), and the like. Network 1103 is for illustration purposes and may be replaced with fewer or additional computer networks. A local area network (LAN) may have one or more of any known LAN topologies and may use one or more of a variety of different protocols, such as Ethernet. Devices 1101, 1105, 1107, 1109 and other devices (not shown) may be connected to one or more of the networks via twisted pair wires, coaxial cable, fiber optics, radio waves or other communication media.

As seen in FIG. 11, computing device 1101 may include a processor 1111, RAM 1113, ROM 1115, network interface 1117, input/output interfaces 1119 (e.g., keyboard, mouse, display, printer, etc.), and memory 1121. Processor 1111 may include one or more computer processing units (CPUs), graphical processing units (GPUs), and/or other processing units such as a processor adapted to perform computations associated with machine learning. I/O 1119 may include a variety of interface units and drives for reading, writing, displaying, and/or printing data or files. I/O 1119 may be coupled with a display such as display 1120. Memory 1121 may store software for configuring computing device 1101 into a special purpose computing device in order to perform one or more of the various functions discussed herein. Memory 1121 may store operating system software 1123 for controlling overall operation of computing device 1101, control logic 1125 for instructing computing device 1101 to perform aspects discussed herein, machine learning software 1127, training set data 1129, and other applications 1131. Control logic 1125 may be incorporated in and may be a part of machine learning software 1127. In other embodiments, computing device 1101 may include two or more of any and/or all of these components (e.g., two or more processors, two or more memories, etc.) and/or other components and/or subsystems not illustrated here.

Devices 1105, 1107, 1109 may have similar or different architecture as described with respect to computing device 1101. Those of skill in the art will appreciate that the functionality of computing device 1101 (or device 1105, 1107, 1109) as described herein may be spread across multiple data processing devices, for example, to distribute processing load across multiple computers, to segregate transactions based on geographic location, user access level, quality of service (QoS), etc. For example, computing devices 1101, 1105, 1107, 1109, and others may operate in concert to provide parallel computing features in support of the operation of control logic 1125 and/or machine learning software 1127.

### SAMC Control of Multiple PCLCs

Healthcare providers are subject to significant task burden while managing critical care patients. Patients in delicate status require continuous adjustment of parameters in medical devices such as ventilators, anesthesia machines, and infusion pumps to meet pre-defined physiological goals. In a variety of applications, including on the future battlefield or other environments in which medical resources and personnel will be scarce and overburdened due to limited evacuation opportunities but increased mass-casualty scenarios, caregivers would benefit from assistance with repetitive, cumbersome tasks to lighten their workload and improve casualty outcomes.

Physiological closed-loop controllers (PCLCs) can unburden critical care staff from the constant adjustment of these devices. PCLCs can provide assistance by performing casualty resuscitation, fluid management, ventilation and/or maintenance of anesthesia, as well as other critical functions, allowing the provider to focus on more cognitively demanding tasks. As these PCLCs are often designed to operate as independent systems, when operating simultaneously on the same patient there is potential for them to perform conflicting actions with each other, possibly resulting in harm to the patient. The synchronous independent operation of all systems can result in conflicts while each individual PCLC strives to achieve a specific physiological goal.

To address this problem, a supervisory algorithm suitable for overseeing individual sub-system PCLCs to maintain harmonization. To prevent conflicts and improve synergy, a supervisory algorithm for overseeing individual sub-system PCLCs in a system and in methodology described herein, is oriented towards the early care of combat casualties. Accordingly, the following goals were achieved: to develop a computer-based expert system capable of coordinating the concurrent (simultaneous) operation of multiple PCLCs on a single patient in a manner that ensures safety and optimizes efficacy of treatments; and to build a knowledge base using existing clinical practice guidelines and input from subject matter experts (SMEs), to be integrated with an inference engine for the management of multiple PCLCs.

Initial logic was developed for each PCLC to individually track specific physiological parameters, such as, for example: (i) the resuscitation and fluid management PCLCs track blood pressure, (ii) oxygen and mechanical ventilation PCLCs track blood oxygen saturation and exhaled carbon dioxide concentration, respectively, (iii) an anesthesia PCLC tracks sedation level, (iv) fluid delivery PCLC, (v) vasopressor therapy PCLC, and (vi) hemorrhage control PCLC. As will be referenced below, any number of physiological parameters may be tracked and controlled. Two primary methods used to develop supervisory algorithm logic include: (i) clinical practice guidelines for the recommended ranges of operation and safety limitations for PCLCs; (ii) clinical subject matter experts (SME) created vignettes involving traumatic injuries to evaluate PCLC responses, followed by SME identification of PCLC conflicts and alternative PCLC actions.

The knowledge gathering exercises identified the need for a supervisory system for oversight over the operation of multiple individual PCLCs and conflict resolution. Logic was collected and may be graphically represented using And-Or Tree diagrams, for example, which subsequently constituted the more than 60 logical rules for the supervisory algorithm. The developed supervisory system may split its logic tree based on three shock severities (mild, moderate, severe per ACLS guidelines), if fluid non-responsive has been identified, and the presence of four complications (pneumothorax, traumatic brain injury, sepsis, prolonged field care) that impact how each sub-system will be managed, for example.

This supervisory system of the SACM allows synergizing the function of multiple independent PCLCs simultaneously, enhancing their clinical effectiveness in a safe manner. This enables the combined system to simultaneously care for multiple casualties while ensuring synchronized clinical intervention. Simulations and interactions with SMEs may be conducted on an on-going basis to identify any gaps in the supervisory logic and validate its ruleset. SACM has therefore demonstrated the ability to hold, process, and facilitate the logic as a supervisory entity for the PCLCs.

An overall goal of the embodiments described herein is to harmonize multiple closed loop controllers under a supervisory management system and method that handles conflicts, changes set points, manages the casualty at a higher level, an important step toward automating trauma care. The use of automated closed loop controllers for fluid infusion, vasopressor administration, mechanical ventilatory management of CO2, oxygen management by a ventilator, anesthesia management, and automated extremity hemorrhage control via tourniquet, etc. are evaluated. Specifics of the rules controlling the algorithm are based on clinical input, internally developed ideas, and clinical practice guidelines for civilian or military care. These are arranged in a rules engine for overseeing each controller which will eventually be tested with a hardware in loop test platform for each of these subsets.

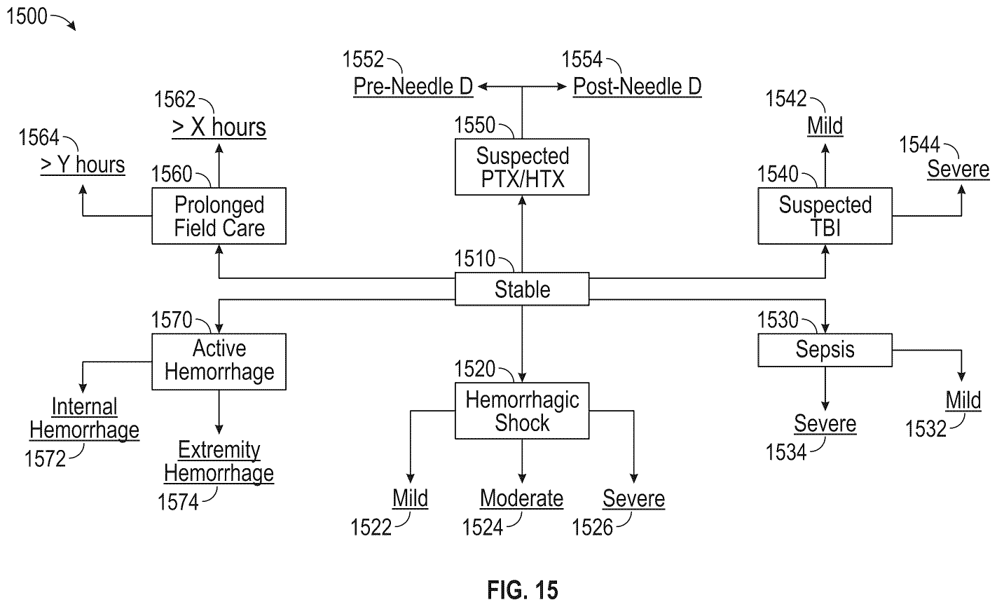

The SACM manages individual PCLCs by monitoring and processing patient vitals, lab values, and determining “casualty state” or combination thereof. As illustrated in a variety of drawings, including FIGS. 15-31, these states may include, for example, stable; hemorrhagic shock (mild, moderate, severe); traumatic brain injury (TBI); prolonged field care (PFC); septic shock (mild, severe); internal hemorrhage; extremity hemorrhage; pre-pneumothorax remedy; and post-pneumothorax remedy. Other casualty states to be monitored and processed may be included.

Therefore, in accordance with additional embodiments presented herein, a system for adaptive supervisory control of physiologic closed-loop controllers includes: a controller in communication with and operable to simultaneously control a plurality of physiological closed-loop controllers (PCLCs) of a corresponding plurality of autonomous devices for automated patient care and to receive in real-time a plurality of physiological state information of a patient detected by the plurality of autonomous devices; and a supervisory algorithm for casualty management (SACM) of the controller that monitors the physiological state information of the patient received by the controller, continuously determines a plurality of casualty states of the patient from the received physiological state information of the patient, and adaptively controls operation of the plurality of physiological closed-loop controllers (PCLCs) responsive to the determined casualty states of the patient, the SACM operable to simultaneously control and adjust operation of the PCLCs responsive to changes in two or more casualty states of the patient to harmonize operation of the PCLCs in accordance with a plurality of supervisory rules of the SACM.

Further, in accordance with these additional embodiments presented herein, a method configured to provide supervisory control of physiologic closed-loop controllers includes: monitoring in real-time physiological state information of a patient and continuously determining from the physiological state information a plurality of casualty states of the patient; and in response to changes in two or more determined casualty states of the patient, simultaneously adaptively controlling and adjusting operation of a plurality of physiological closed-loop controllers (PCLCs) in accordance with a plurality of supervisory rules of a supervisory algorithm for casualty management (SACM) to harmonize operation of the PCLCs.

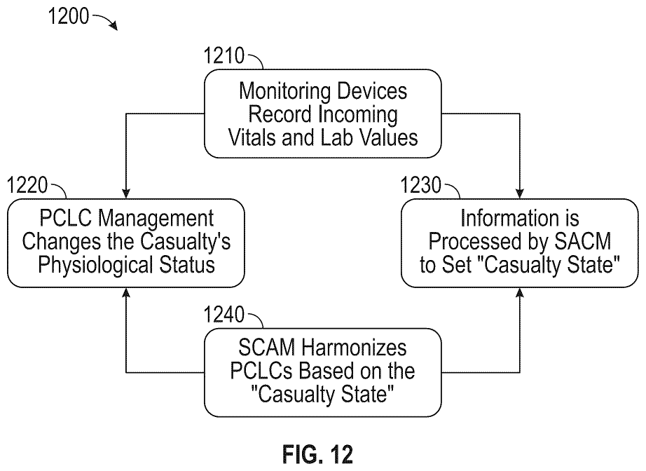

Referring now to FIG. 12, a flow diagram 1200 of SACM, casualty state classification and PCLC interaction is illustrated. At block 1210, monitoring devices record incoming vitals and lab values of the patient; these monitoring devices are autonomous devices for automated patient care operable to detect in real-time physiological state information of a patent. For example, such autonomous devices may control fluid administration, vasopressor therapy, mechanical ventilation, oxygenation management, hemorrhage control, and anesthesia management of the patient. These vital and/or lab values may include a variety of measurable physiological values of a patient, including without limitation, blood pressure, systemic vascular resistance (SVR), fluid responsiveness, fluid infusate type, cuff pressure, target cuff pressure, oxygen saturation (SpO2), target oxygen saturation (SpO2), end-tidal carbon dioxide (EtCO2), target EtCO2, current mean arterial pressure (MAP) or cardiac output (CO), hemorrhage status, shock status, spontaneous (current) respiratory rate (RR), Glascow Coma Scale (GCS), positive end expiratory pressure (PEEP), vasopressor-ARC (V-ARC), patient weight estimate, lab results (Hematocrit), BIS or EEG readings, anesthetic type used, target anesthetic depth, flow rate factor, extremity bleed status, etc. These values can be inputs or outputs to the SACM and/or respective PCLCs controlled by the SACM, and may additionally be provided by a user (operator) of the SACM and/or PCLCs.

At block 1220, PCLC management can change the casualty physiological status based upon the information from block 1210, while at block 1230 the information from block 1210 is processed by SACM to set the current “casualty state(s)” of the patient. At block 1240, the SCAM harmonizes the PCLCs based upon the determined “casualty state(s)” determined at block 1230.

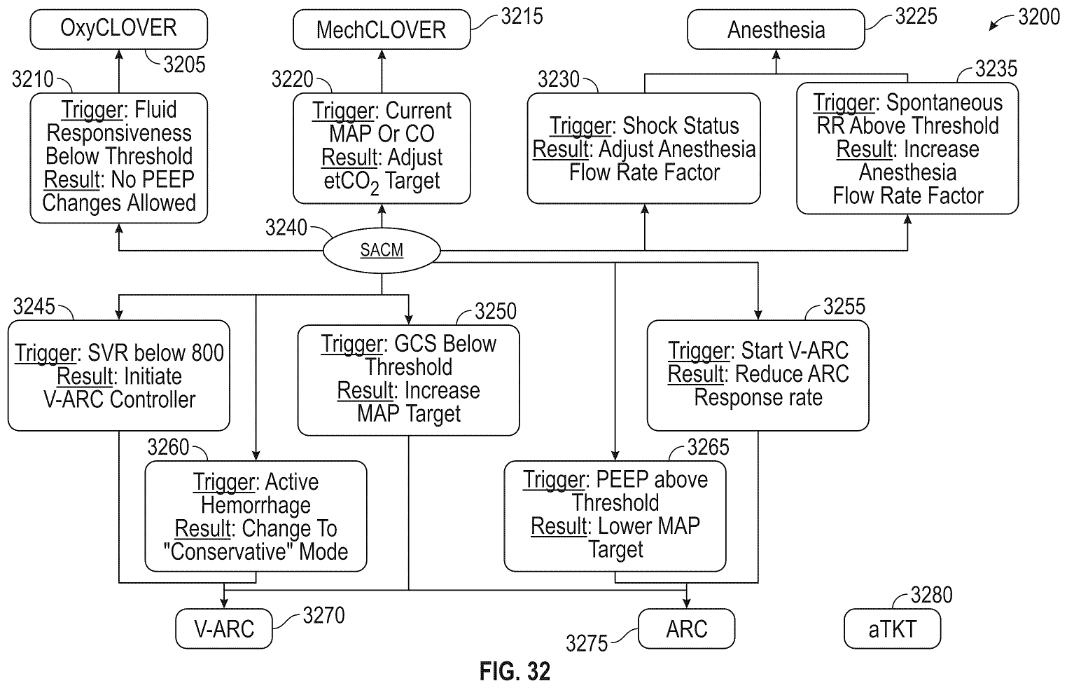

Consider the following examples for various PCLCs, which provide an example summary of individual PCLC logic with respective inputs and outputs for control function. A fluid administration PCLC controls fluid delivery based on fluid responsiveness measurement and pressure rate increase. Inputs to a fluid administration PCLC may therefore be current blood pressure measurements, target pressure, and resuscitation aggressiveness and the output may be pump flow rate, which is provided to the SACM simultaneously controlling the fluid administration PCLC as well as other PCLCs. A vasopressor therapy PCLC may be a fuzzy logic controller responsible for tracking target pressure error and rate of error change for norepinephrine (NE) infusion. Its inputs may include blood pressure, target pressure, dosage aggressiveness and activation aggressiveness and its output may be pump NE flow rate provide to the SACM. A mechanical closed loop ventilator (MechClover) PCLC may be a decision table-based controller for managing end-tidal carbon dioxide. Its input may be a target EtCO2, and it may produce tidal volume and respiratory rate (RR) as its outputs. A oxygen closed loop ventilator (OxyClover) PCLC for oxygenation ventilation management may likewise be a decision table-based controller for managing blood oxygen saturation levels. Its input may be a target SpO2 and its outputs to the SACM may be fraction of inspired oxygen or oxygen content (FiO2) and peak inspiratory pressure (PIP). A hemorrhage control PLCL may be an automated extremity pneumatic tourniquet for maintaining hemorrhage control. Its inputs may accordingly be cuff pressure and a status of deployed or retracted. The output to the SACM may be a status of extremity hemorrhage. An anesthesia management PCLC may be a preliminary Ketamine-based controller with feedback related to casualty motion. Inputs may include a patient's level of voluntary movement and outputs to the SAMC may include Ketamine and infusion rate, for example. Further examples of individual PCLC logic with respective inputs and outputs for control function are further illustrated in the discussion of FIG. 32.

System rules for SACM were developed by (i) searching clinical practice guidelines for the recommended operation and safety limitations for PCLCs, and (ii) consulting with clinical SMEs to create vignettes involving traumatic injuries against which the systems were tested. The SMEs evaluated the responses of the PCLCs, conflicts and opportunities to improve synergy were addressed by adjusting existing supervisory rules or adding necessary ones to bridge gaps found in the logic.

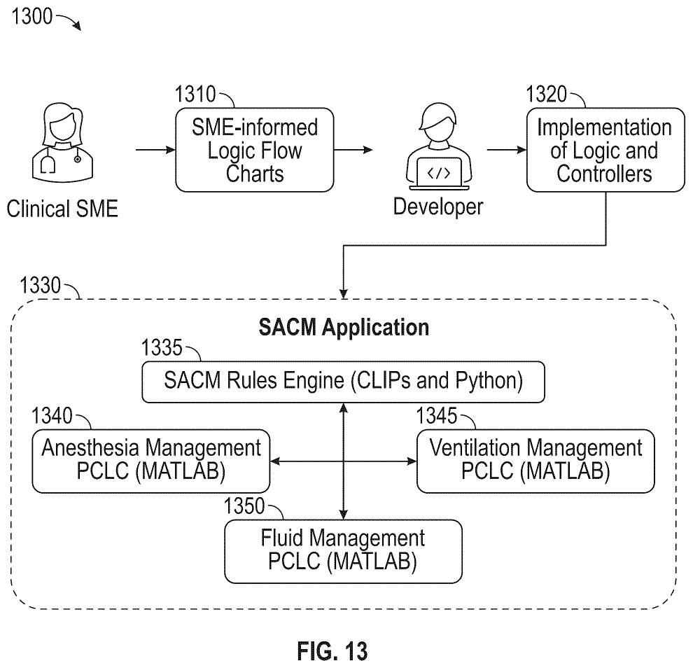

Referring now to FIG. 13, a flow diagram 1300 of SACM development is illustrated. The knowledge base devised in collaboration with the clinical SMEs was encoded as a set of logical rules using the CLIPS programming language, which are executed by the CLIPS Rules Engine. In accordance with an example embodiment, as PCLCs are developed in MATLAB, a software interface was also established to interact with that platform shown in FIG. 13, allowing the Rules Engine to receive setpoint values, parameters from the PCLCs, and patient physiological measurements to adjust settings as dictated by the knowledge base. At block 1310, SME-informed logic flow charts are based upon a review from a SME, such as a clinical SME, that are provided to a developer that creates an implementation of logic and PCLC controllers that are coordinated and controlled by the SAMC, at block 1320. Within the SAMC application 1330, the SACM rules engine 1335, which may be implemented using CLIPs and Python or other rules engines and programming languages, interfaces and controls operation of one or more PCLCs 1340, 1345, 1350, which in this example are an anesthesia management PCLC 1340, a ventilation management PCLC 1345, and a fluid management PCLC 1350 as shown.

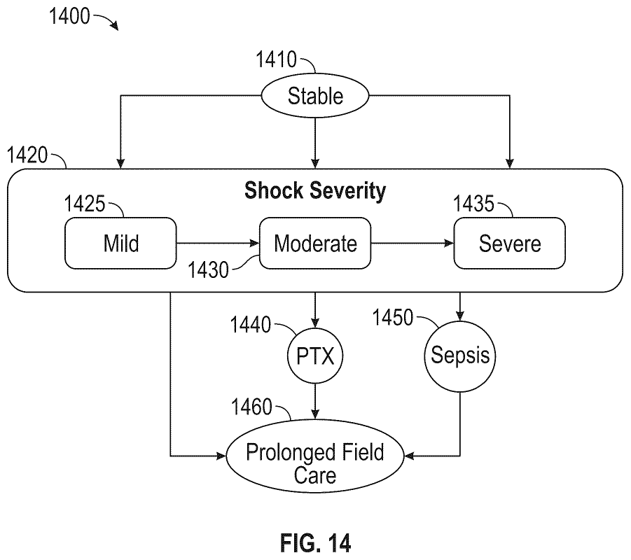

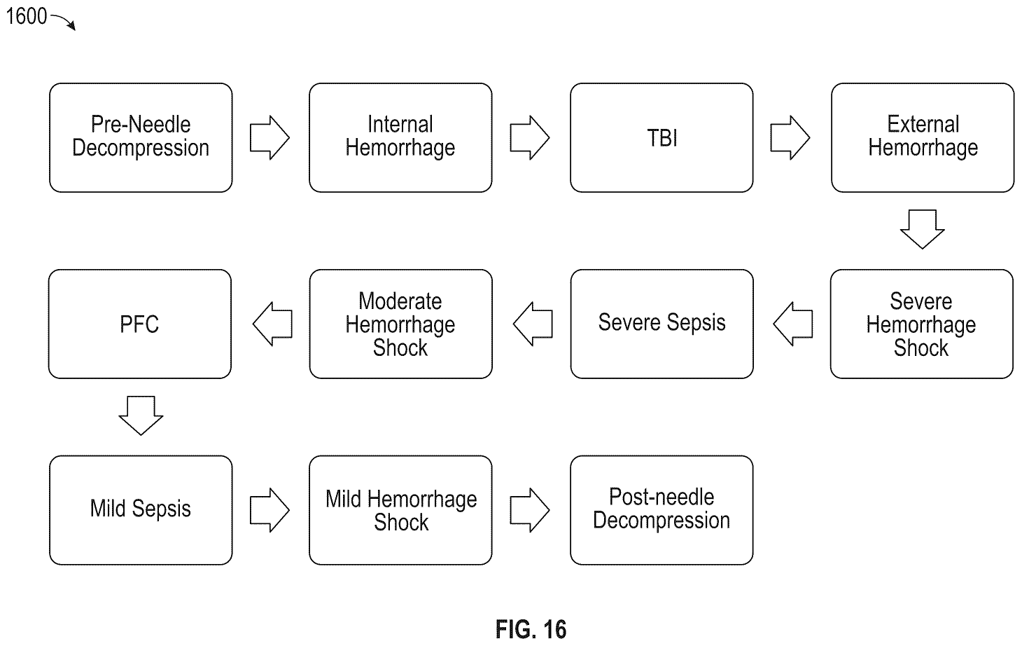

FIGS. 14 and 15 illustrate example SACM state diagrams with respect to various casualty states. In the example state diagram flow 1400 of FIG. 14, a number of casualty states are shown, including stable 1410; shock severity 1420 with mild, moderate and severe degrees of shock severity 1425, 1430, 1435, respectively; pneumothorax (PTX) 1440; Sepsis 1450; and PFC 1460. FIG. 15 provides another example state diagram flow 1500 showing a number of casualty states with potential trigger logic shown. Included in this example are stable casualty state 1510; hemorrhagic shock casualty state 1520 with respective triggers mild 1522, moderate 1524, and severe 1526 degrees of hemorrhagic shock; sepsis casualty state 1530 with triggers mild 1532 and severe 1534 degrees of sepsis severity; suspected TBI casualty state 1540 with triggers mild 1542 and severe 1544 degrees of suspected TBI; suspected PTX/hemothorax (HTX) casualty state 1550 with triggers pre-needle decompression (Pre-needle D) to treat a tension pneumothorax or a post-procedure care and management of a patient after treatment for a tension pneumothorax (Post-needle D) 1554; PFC casualty state 1560 with triggers that may be greater than X hours 1562 or greater than Y hours 1564; active hemorrhage casualty state 1570 with triggers that may be an internal hemorrhage 1572 or an extremity hemorrhage 1574, as shown. Referring to FIG. 16, an example casualty state triage flow that may be employed by the SACM is shown. Other triage flows may be used without departing from the scope of the disclosure.

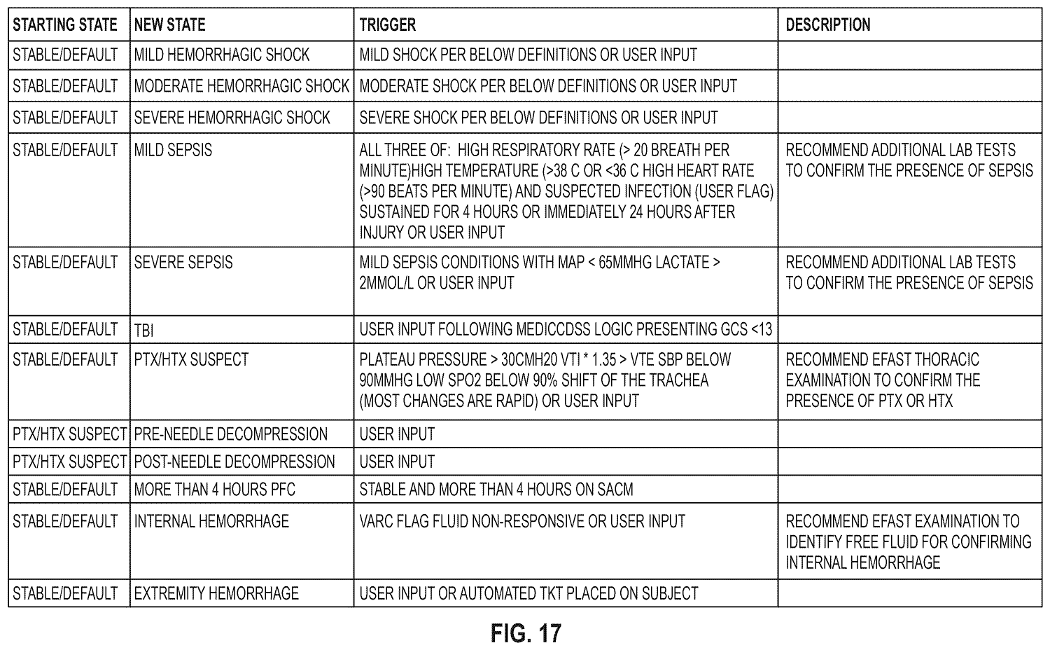

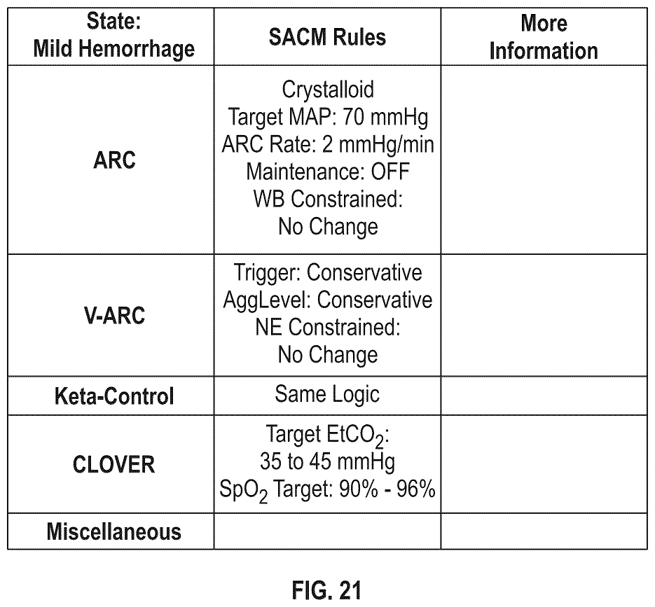

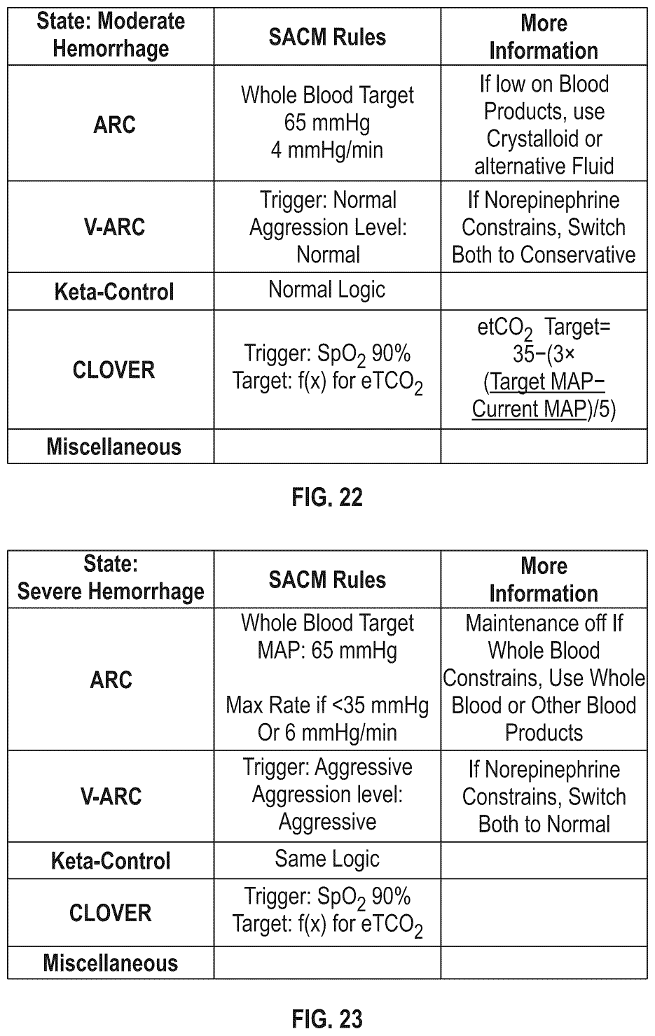

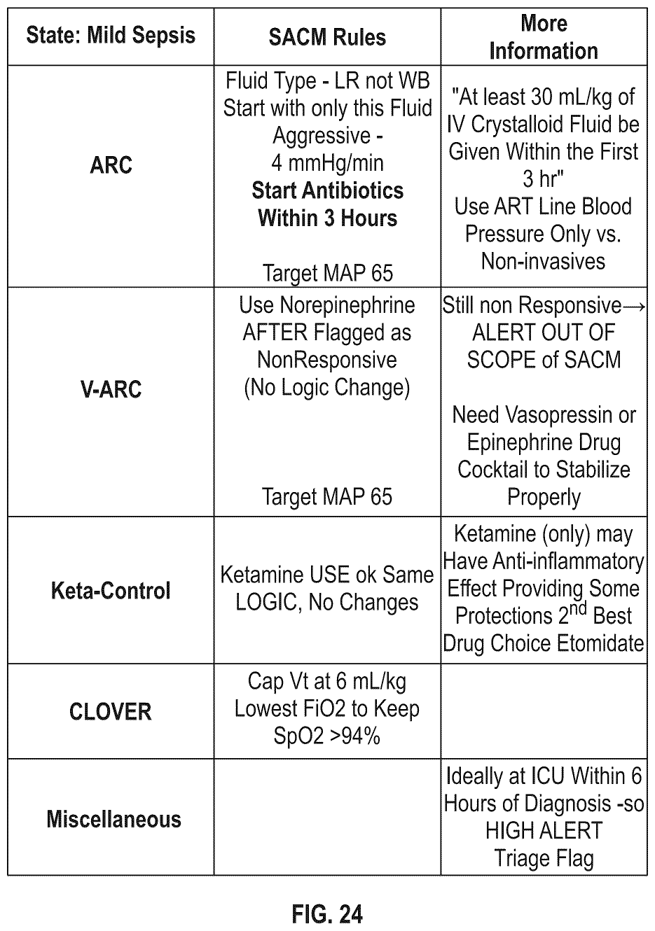

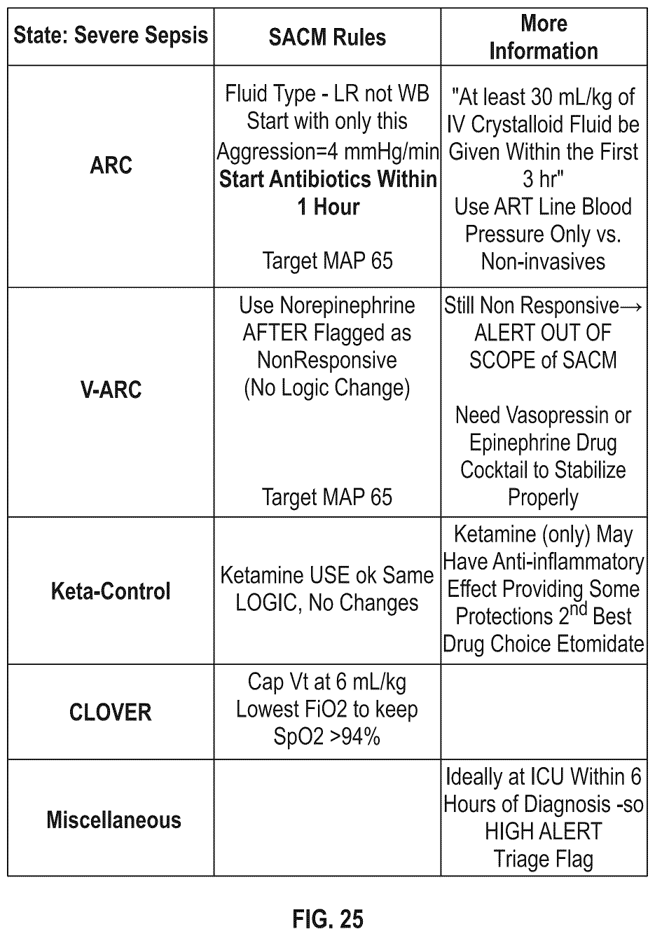

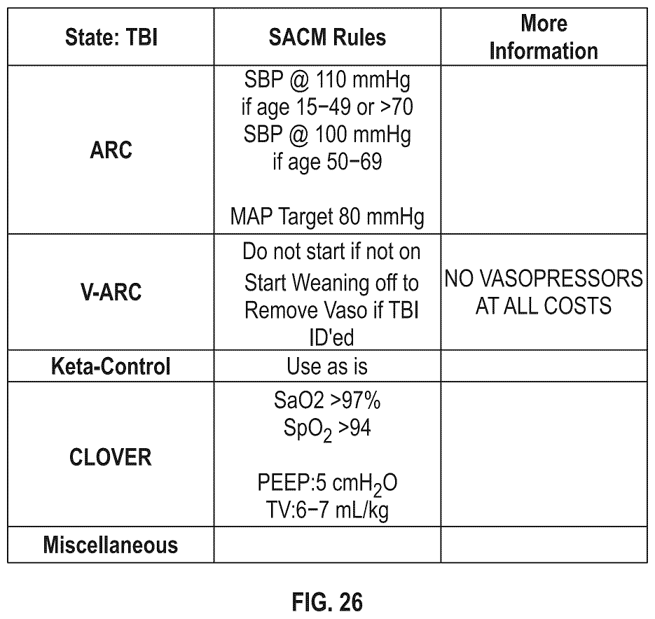

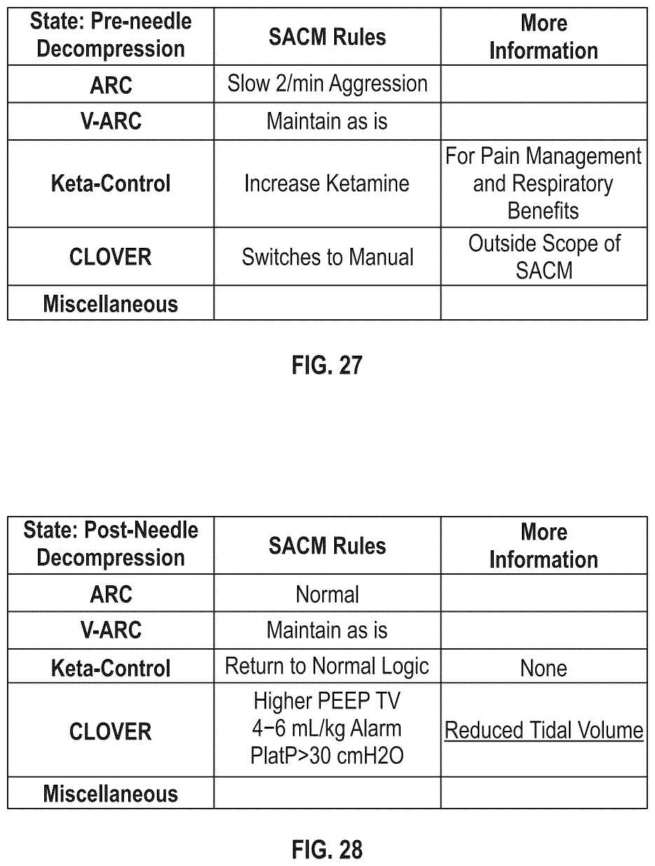

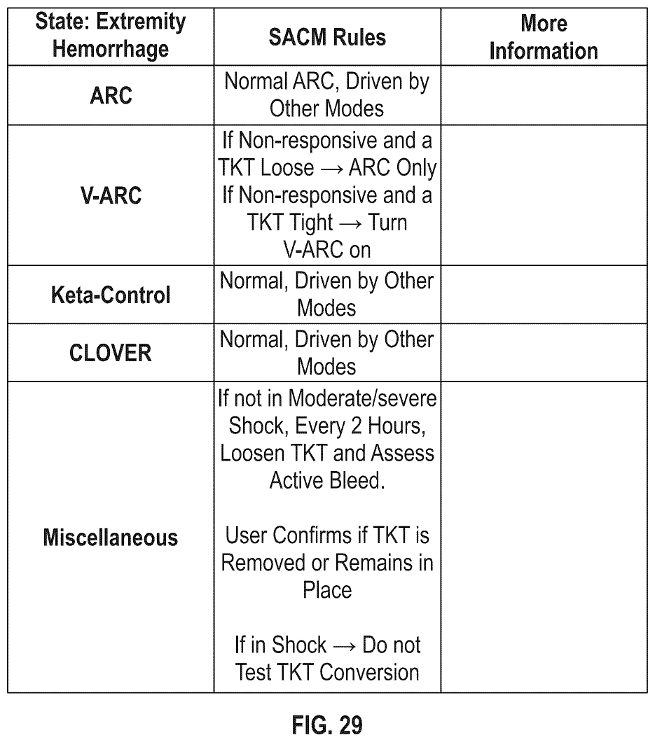

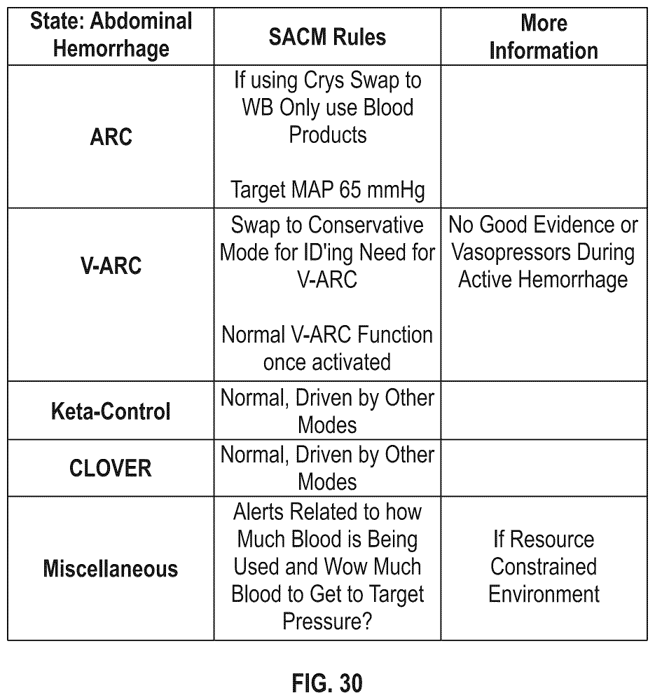

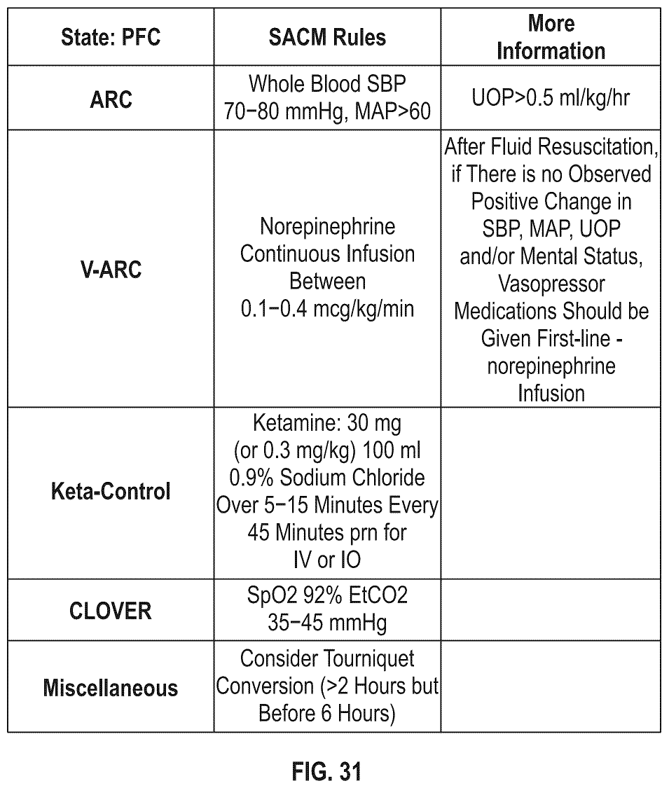

Referring to FIG. 17, examples of various stable or default casualty state to subsequent, new casualty state progressions are shown, together with potential trigger conditions. Again, these progressions may be different or may change as needed and may be informed by updated or improved information from a SME or SAMC logic or even user input. For example, as shown in the first entry of the table of FIG. 17, a new casualty state of Mild hemorrhagic shock may be triggered by the definitions for mild hemorrhagic shock definitions (see as an example the logic table for Mild Hemorrhage and SACM rules for various PCLCs thereof in FIG. 21). In the second entry of the table of FIG. 17, a new casualty state of Moderate hemorrhagic shock may be triggered by the definitions for moderate hemorrhagic shock definitions (see as an example the logic table for Moderate Hemorrhage and SACM rules for various PCLCs thereof in FIG. 22). In the third entry of the table of FIG. 17, a new casualty state of Severe hemorrhagic shock may be triggered by the definitions for severe hemorrhagic shock definitions (see as an example the logic table for Severe Hemorrhage and SACM rules for various PCLCs thereof in FIG. 23). In the fourth entry of the table of FIG. 17, a new casualty state of mild sepsis from a stable or default casualty state may be triggered by various definitions, some example definitions of which are shown (see also as an example the logic table for Mild Sepsis and SACM rules for various PCLCs thereof in FIG. 24). In the fifth entry of the table of FIG. 17, a new casualty state of severe sepsis from a stable or default casualty state may be triggered by various definitions, some example definitions of which are shown (see as an example the logic table for Severe Sepsis and SACM rules for various PCLCs thereof in FIG. 25). In the sixth entry of the table of FIG. 17, a new casualty state of traumatic brain injury TBI from a stable or default casualty state may be triggered by various definitions, some example definitions of which are shown (see also as an example the logic table for TBI and SACM rules for various PCLCs thereof in FIG. 26). In the seventh entry of the table of FIG. 17, a new casualty state of PTX/HTX suspected from a stable or default previous casualty state may be triggered by various definitions, some example definitions of which are shown. In the eighth and ninth entries of the table of FIG. 17, new casualty states of pre-needle decompression and post-needle decompression from a previous state of PTX/HTX suspected are shown; the triggers are user inputs following the pre-/post-needle decompressions as shown. See also FIGS. 27 and 28 that illustrate example logic tables for pre-needle decompression and post-needle decompression logic tables and SACM rules for various PCLCs. The tenth entry of FIG. 17 shows a new casualty state of more than four (4) hours PFC from a table or default previous casualty state, in which the trigger is a patient that is stable and more than four (4) hours on the SACM (see also as an example the logic table for PFC and SACM rules for various PCLCs thereof in FIG. 31). The eleventh and twelfth entries of FIG. 17 show movement from a stable/default casualty state to an internal hemorrhage and an extremity hemorrhage, respectively. For the new internal hemorrhage casualty state the trigger may be V-ARC flag fluid showing non-responsive or user input, for example. For an extremity hemorrhage, the trigger may be user/operator input or an automated tourniquet placed on the patient. FIGS. 29 and 30 are example logic tables for an extremity hemorrhage and an internal (abdominal) hemorrhage, respectively, and SACM rules for various PCLCs thereof.

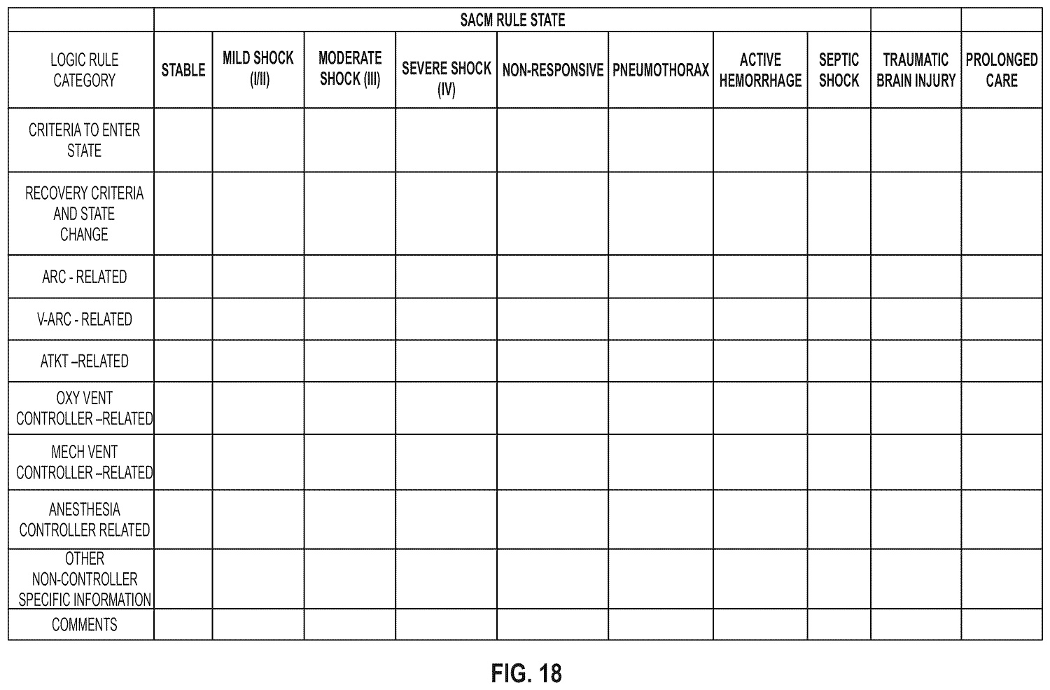

FIG. 18 is an example SACM preliminary logic table. The SACM Rule State columns across the top are the following example rule states: stable, mild shock, moderate shock, severe shock, non-responsive, pneumothorax, active hemorrhage, septic shock, TBI, and prolonged care PFC. The rows across the logic table may include various logic rule categories. In the specific example of FIG. 18, these may include: criteria to enter state, recovery criteria and state change, ARC-related, V-ARC related, aTKT-related, OxyVent (an oxygen ventilation/management, like OxyClover), Mech Vent (a mechanical ventilation/management, like MechClover), anesthesia controller-related, other non-controller specific information. More or less of these columns and rows are be employed in such a SACM logic table within the scope of the disclosure.

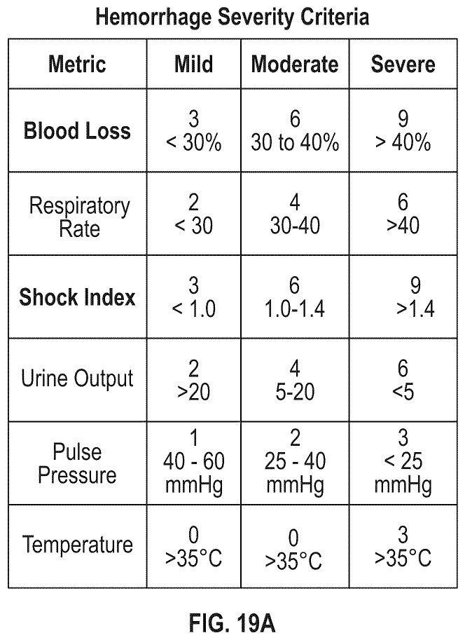

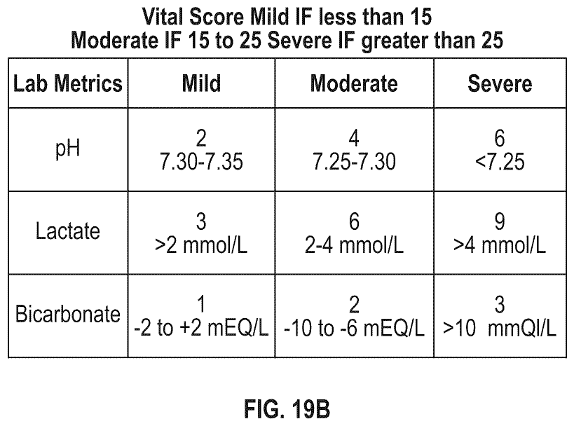

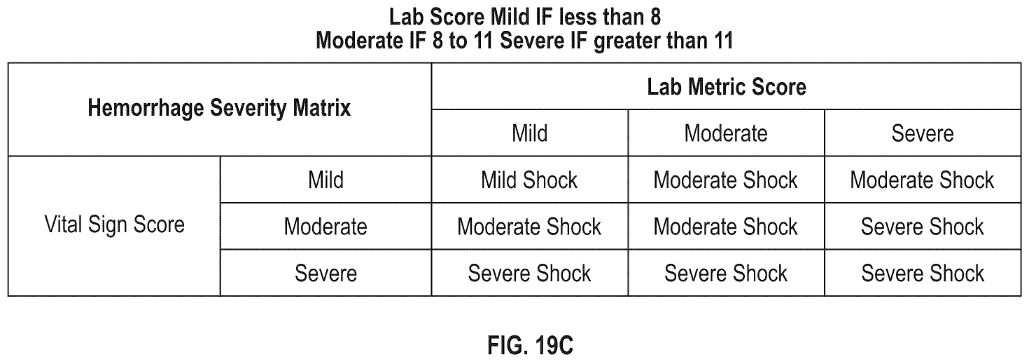

Referring now to FIGS. 19A-19C, example information with respect to hemorrhage casualty states is provided. In FIG. 19, hemorrhage severity criteria is provided, showing various metrics for mild, moderate and severe hemorrhage casualty states. FIG. 19B provides example vital scores for mild, moderate and severe hemorrhage casualty states. Initial hemorrhage severity may be based on vitals alone or lab results if available. Vitals scoring may be based on urine output, estimated blood loss, shock index, pulse pressure, respiratory rate and temperature. Lab results aggregate values for lactate, blook pH and bicarbonate levels as shown in FIG. 19B. An overall scoring system for mild, moderate, severe hemorrhage shock that may be employed is shown in the table of FIG. 19C.

By way of example and not limitation, the following are examples of criteria to enter state for each of these example casualty states include:

Stable casualty state: Default State, No Criteria Mild shock casualty state:

### IF

Percent Estimated Blood Loss<30%
USER Hemorrhage FLAG TRUE
Heart Rate<120 BPM
Blood Pressure Normal (MAP>80 mmHg; Systole>110 mmHg)
Presenting Respiratory Rate<30 BPM
Urine Output>20 mL/hr
   Moderate shock casualty state:

### IF

Percent Estimated Blood Loss 30-40%
USER Hemorrhage FLAG TRUE
Heart Rate 120 to 140 BPM.
Blood Pressure Low (MAP 80 to 35 mmHg)
Presenting Respiratory Rate<30 to 40 BPM
Urine Output 20 to 5 mL/hr
   Severe shock casualty state:

### IF

Percent Estimated Blood Loss>40%
USER Hemorrhage FLAG TRUE
Heart Rate>140 BPM.
Blood Pressure Very Low (MAP<35 mmHg)
Presenting Respiratory Rate>35 BPM
Urine Output<5 mL/hr
   Non-responsive casualty state:

### IF

ARC Non-Response flag TRUE
Clinician flag Non-Responsive TRUE
Systemic Vascular Resistance<800 dynes/scm⁵
Fluid overload flag TRUE
Poor Organ Perfusion flag TRUE
ARC Systolic Drop>30 mmHg (120 to 90 mmHg)

### THEN

eFAST Exam Recommended
MARCH evaluation Recommended
aTKT Bleed Rate Check
Sepsis “assessment” recommended

### FINAL IF

Evaluation Criteria Negative
   Pneumothorax casualty state:

### IF

Rapid increase in peak airway pressure>50 cm H2O
Rapid drop in tidal volume
Hypoxemia sudden
Hypotension (≤60 MAP) quick development

### OR

Clinician Flag TRUE (pre-existing dx of pneumothorax)

### THEN

Recommend pneumothorax examination (check for breath sounds on bilateral chest OR US exam OR radiograph)

### IF Positive

Recommend chest tube placement and initiate ARDSnet ventilation Active hemorrhage casualty state:

### IF

Clinician flag TRUE “bleed”

### OR IF

Non-response flag TRUE

### THEN

Bleed evaluation recommended
   Septic shock casualty state:
Conduct NEWS2 Diagnostic

### IF

Score of 5 or greater

### THEN

Trigger alarm for clinician's assessment (eFAST exam to confirm no internal hemorrhage)
Trigger flag to clean wound (and swab/get a blood draw to start cultures)
Adjust frequency of NEWS2 assessment from every hour to every 15-30 minutes TBI casualty state:

### IF

Patient has a “presenting” GCS score of≤13

### OR

Clinical flag TBI risk AND shock flag TRUE
For severely brain injured patients, immediate intubation with adequate ventilation (PaCO2 of 35) and oxygenation and restoration of intravascular volume are the most critical first line treatment for a severely head injured patient

### THEN

Patient is managed in a monitored setting hourly
Avoid cerebral hypoxia by maintaining the PaO2>80 mmHg or oxygen saturation >93%
Avoid cerebral hypoperfusion by keeping SBP>90 mmHg PFC casualty state:
   Revert to stable state/mild hemorrhage while watching amount of fluid given
Fluid overload prevention (AKI, TRALI)
Lung protective ventilation (Vent-induced lung injury)
Thrombosis
   Additional concerns with PFC (aside from sepsis)
Vent-induced lung injury, giving the patient ARDS—prevention: lung protective ventilation
TRALI (transfusion related acute lung injury)—can damage the lung by giving too much fluids
Fluid overload—AKI or fluid dilution (26% trauma patients develop AKI within first 28 days)
VTE/PE—looking at a hemorrhage model of injury, but if moving to a more prolonged model increases the risk of a thrombotic event

By way of example and not limitation, the following are examples of recovery criteria and state change for each of these example casualty states include:
Stable casualty state: N/A
Mild shock casualty state: SpO2>94%, HR between 60 and 100 BPM, Urine Output>0.5 mL/kg/hr, Return to Stable State
Moderate shock casualty state: MAP>95% Target MAP, 10 minutes and ARC mean infusion rate<Threshold, 10 minutes, Return to Mild Shock State
Severe shock casualty state: MAP<35 mmHg, Return to Moderate Shock State
Non-responsive casualty state: Vaso Rate at 0 mL/hr for 10 minutes and MAP>95% Target MAP, 10 minutes, Return to Prior State
Pneumothorax casualty state: SpO2>90% for 10 minutes, MAP>95% target for 10 minutes, Flag TRUE that chest tube was placed, Return to Prior State
Active hemorrhage casualty state: MAP>95% target for 30 minutes, Heartrate between 60-100 BPM, Return to Prior State
Septic shock casualty state: Score<5: Score 5-6: Score>6: Blood marker? Lactate levels? Lactate should be high given the injury state. Blood culture if sepsis is indicated. Treat with broad spectrum empirics followed by narrow when culture is received from the lab.
TBI casualty state: GCS≥14 OR CANNOT Leave state once flagged TBI PFC casualty state: N/A

By way of example and not limitation, Arc-related rule states for each of these example casualty states include:

Stable casualty state: Basal Rates only.

### 4-2-1 Rule

$$> 20 kg = 60 mL + 1 mL / kg / hr$$

$$10 < 20 kg = 40 mL + 2 mL / kg / hr$$

$$< 10 kg = 4 mL / kg / hr$$

Do ×1.5 for austere environments but have to take fluid overload into consideration (including risk of infection and poor outcomes).

Mild shock casualty state:
IF head injury—target MAP 80 mmHg, ELSE—target MAP 65 mmHG
Recommended Fluid Infusate: Crystalloid
   Moderate shock casualty state:
   IF head injury—target MAP 80 mmHg, ELSE—target MAP 65 mmHG

### Recommended Fluid Infusate

Whole Blood
1:1:1 Plasma: RBCs: Platelets
1:1 Plasma RBCs
Plasma only
RBCs only
Crystalloid
IF resource constrained FLAG TRUE, ask amount of blood produced and ALERT when LOW
   Severe shock casualty state:
   IF head injury—target MAP 80 mmHg, ELS—target MAP 65 mmHG

### Recommended Fluid Infusate

Whole Blood
1:1:1 Plasma: RBCs: Platelets
1:1 Plasma RBCs
Plasma only
RBCs only
Crystalloid
IF resource constrained FLAG TRUE, ask amount of blood produced and ALERT when LOW
   Non-responsive casualty state:
   IF head injury—target MAP 80 mmHg, ELSE—target MAP 65 mmHG

### Recommended Fluid Infusate

Crystalloid
ARC Mode shifts to
ARC off, basal rate only
ARC slowed response rate
ARC normal function
   Pneumothorax casualty state: support the pressure to prevent crashing while chest tube is placed
   Active hemorrhage casualty state:
   IF head injury—target MAP 80 mmHg, ELSE—target MAP 65 mmHG

### Recommended Fluid Infusate

Whole Blood
1:1:1 Plasma: RBCs: Platelets
1:1 Plasma RBCs
Plasma only
RBCs only
Crystalloid.
IF resource constrained FLAG TRUE, ask amount of blood produced and ALERT when LOW
   Septic shock casualty state:
IF score is>5
IF goal MAP>65 mmHg is not attained with fluid resuscitation, the initiation of vasopressors is indicated.
THEN Cefazolin 2 g in 250 mL NS IV over 5 minutes every 8 hours for 24 hours I adequate for most dirty wounds of the head and neck, torso, and extremities.
TBI casualty state: target MPA of 80 mmHg as the goal of management is to maintain a CPP of >60 mmHg
PFC casualty state: N/A

By way of example and not limitation, V-ARC-related rule states for each of these example casualty states include:
Stable casualty state: System OFF
Mild shock casualty state: System OFF
Moderate shock casualty state: System OFF
Severe shock casualty state: System OFF
Non-responsive casualty state: System ON, use Norepinephrine vasopressor infusion rate >threshold (0.3 mcg/kg/hr)→ALARM
Pneumothorax casualty state: N/A
Active hemorrhage casualty state: N/A
Septic shock casualty state: System ON, use Norepinephrine vasopressor infusion rate>threshold (0.3 mcg/kg/hr)→ALARM
TBI casualty state:

If SBP remains less than 100-110 mmHg despite appropriate resuscitation and hemorrhage control, a vasopressor agent should be started if available
Norepinephrine continuous infusion 0.1-0.4 mcg/kg/min.
Vasopressin continuation infusion 0.01-0.04 units PFC casualty state: N/A

By way of example and not limitation, aTKT tourniquet-related rule states for each of these example casualty states include:
Stable casualty state: Either OFF, or ON. ALARM if bleed detected.
Mild shock casualty state: Either OFF, or ON. ALARM if bleed detected.
Moderate shock casualty state: Either OFF, or ON. ALARM if bleed detected.
Severe shock casualty state: Either OFF, or ON. ALARM if bleed detected.
Non-responsive casualty state: Either OFF, or ON. ALARM if bleed detected.
Pneumothorax casualty state: N/A
Active hemorrhage casualty state: Tourniquet and Flag/Alert if placed for >2 hours.
Septic shock casualty state: N/A
TBI casualty state: N/A
PFC casualty state: Flag/Alert if torniquet placed for >2 hours

By way of example and not limitation, Oxy Vent-related rule states for each of these example casualty states include:
Stable casualty state: Normal logic
Mild shock casualty state: Normal logic
Moderate shock casualty state: Normal logic
Severe shock casualty state: Normal logic
Non-responsive casualty state: No PEEP change allowed without clinician approval “ALARM”
Pneumothorax casualty state: N/A
Active hemorrhage casualty state: N/A
Septic shock casualty state: N/A
TBI casualty state: Avoid vasoconstriction or vasodilation by maintaining the PaCO₂ between 35 and 40 mmHg. Correlation to EtCO2.
PFC casualty state: N/A

By way of example and not limitation, Mech Vent-related rule states for each of these example casualty states include:
Stable casualty state: etCO2 target at 35 mmHg
Mild shock casualty state: etCO2 target at 35 mmHg

$$Moderate shock casualty state : et CO 2 target at 35 mmHg - ( 3 \times ( \frac{Target Map - Current MAP}{5} ) ) )$$

$$Severe shock casualty state : et CO 2 target at 35 mmHg - ( 3 \times ( \frac{Target Map - Current MAP}{5} ) ) )$$

$$Non - responsive casualty state : et CO 2 target at 35 mmHg - ( 3 \times ( \frac{Target Map - Current MAP}{5} ) ) )$$

Pneumothorax casualty state: N/A
Active hemorrhage casualty state: N/A
Septic shock casualty state: N/A
TBI casualty state: N/A
PFC casualty state: N/A

By way of example and not limitation, anesthesia-controller-related rule states for each of these example casualty states include:
Stable casualty state: Anesthesia Initial Factor=1, Spontaneous RR>Threshold, Factor+0.1
Mild shock casualty state: Anesthesia Initial Factor=1, Spontaneous RR>Threshold, Factor+0.1
Moderate shock casualty state: Anesthesia Initial Factor=0.50, Spontaneous RR>Threshold, Factor+0.1
Severe shock casualty state: Anesthesia Initial Factor=0.25, Spontaneous RR>Threshold, Factor+0.1
Non-responsive casualty state: Anesthesia Initial Factor=Follow prior status, Spontaneous RR>Threshold, Factor+0.1
Pneumothorax casualty state: N/A
Active hemorrhage casualty state: N/A
Septic shock casualty state: N/A
TBI casualty state: Propofol preferred
PFC casualty state: N/A

By way of example and not limitation, other non-controller specific information rule states for each of these example casualty states include:
Stable casualty state: N/A
Mild shock casualty state: N/A
Moderate shock casualty state: N/A
Severe shock casualty state: N/A
Non-responsive casualty state: N/A
Pneumothorax casualty state: ALERT for higher triage priority
Active hemorrhage casualty state: ALERT for active bleed, ALERT for high triage priority
Septic shock casualty state: ALERT for higher triage priority
TBI casualty state: ALERT for higher triage priority
PFC casualty state: N/A

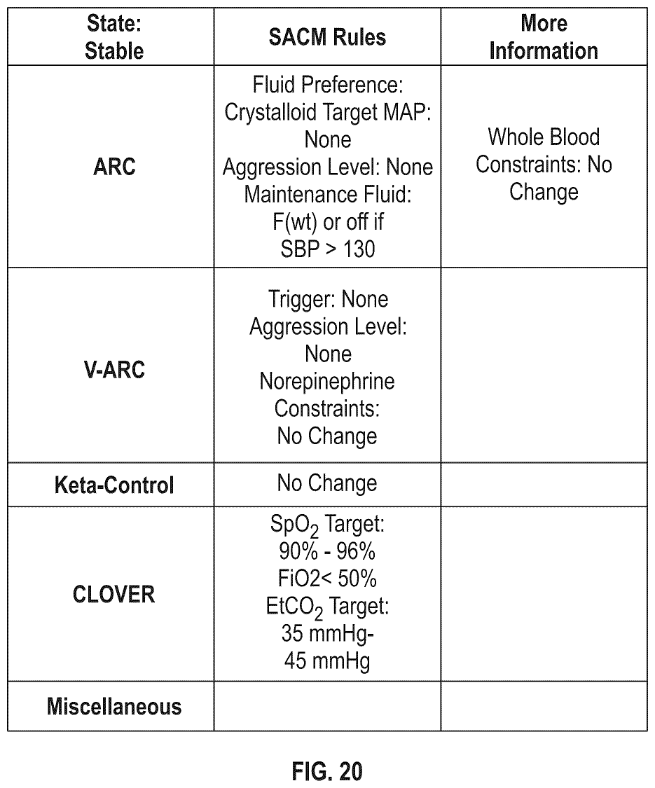

Referring to FIG. 20, an example logic table for a Stable casualty state and SACM rules thereof for various PCLCs are illustrated. Referring to FIG. 21, an example logic table for Mild Hemorrhage casualty state and SACM rules thereof for various PCLCs are illustrated. FIG. 22 is an example logic table for Moderate Hemorrhage casualty state and SACM rules for various PCLCs thereof. FIG. 23 is an example logic table for Severe Hemorrhage casualty state and SACM rules for various PCLCs thereof. FIG. 24 is an example logic table for Mild Sepsis casualty state and SACM rules for various PCLCs thereof. FIG. 25 is an example logic table for Severe Sepsis casualty state and SACM rules for various PCLCs thereof. FIG. 26 is an example logic table for TBI casualty state and SACM rules for various PCLCs thereof. FIGS. 27 and 28 illustrate example logic tables for pre-needle decompression and post-needle decompression new casualty states and SACM rules for various PCLCs thereof. FIGS. 29 and 30 illustrate example logic tables for an extremity hemorrhage and an internal (abdominal) hemorrhage casualty states, respectively, and SACM rules for various PCLCs thereof. FIG. 31 is an example logic table for PFC casualty state and SACM rules thereof for various PCLCs.

Referring now to FIG. 32, block diagram 3200 illustrates a system in which a SACM 3240 is in communication with and controls operation of a number of PCLCs, 3205, 3215, 3225, 3270, 3275 and 3280. Each of the PCLCs is controlled by the SACM in accordance with one or more supervisory rules that may take into account triggering events and results that are set forth in accordance with sets of logical rules of a rules engine of the SACM. Thus, in this particular example, OxyClover PLCL 3205 is controlled by logic block 3210; MechClover 3215 is controlled by logic block 3220; Anesthesia PCLC 3225 is controlled by one or more of logic blocks 3230 and 3235 as shown. V-ARC PCLC 3270 is controlled by one or more of logic blocks 3245, 3250, 3260; ARC PCLC 3275 is controlled by logic blocks 3250, 3255, and 3265, as shown. Additional PCLC aTKT (tourniquet) control may also be controlled by the SACM via one or more logic control blocks, now shown.

Inputs for the various components may be as follows. SACM 3240 have include the following inputs: cardiac output (CO); blood pressure (BP); Glascow Coma Scale (GCS); systematic vascular resistance (SVR); fluid responsiveness (from ARC 3275); shock status; active hemorrhage status; PEEP from an oxygen ventilation system such as OxyClover 3205; spontaneous respiratory rate from a mechanical ventilation system such as MechClover 3215 or MechVent; subject weight estimate; subject (patient) weight estimate; and lab results (Hematocrit).

User inputs, such as from an operator of the SACM system and/or a PCLC of interest, may occur for one or more conditions, including: for low fluid responsiveness (may be an eFAST decision); for an active hemorrhage=flow rate override; for a controlled hemorrhage; for a systematic vascular resistance (SVR) condition; for a fluid responsiveness condition, such as indicated by the ARC; for a defined shock status; for an active hemorrhage status; responsive to PEEP from an oxygen ventilation system such as OxyClover 3205; for a spontaneous respiratory rate from a mechanical ventilation system such as MechClover 3215 or Mech Vent; a subject (patient) weight estimate; and lab results (Hematocrit). V-ARC inputs for V-ARC 3270 may include fluid responsiveness from ARC 3275; blood pressure; target mean arterial pressure (MAP) and controller mode (aggressive or conservative). ARC inputs for ARC 3275 may include blood pressure, target MAP; and fluid infusate type. aTKT inputs for aTKT 3280 may include cuff pressure and target cuff pressure. OxyClover inputs for OxyClover 3205 may include oxygen saturation (SpO2) and target SpO2. MechClover inputs for MechClover 3215 may include EtCO2 and target EtCO2. ACL inputs for Anesthesia 3225 may include VIS or EEG readings, anesthetic type used, target anesthetic depth and flow rate factor.

Triggers for the respective PCLCs may be as follows. V-ARC triggers for V-ARC PCLC 3270 ay include low SVR and fluid non-responsiveness. ARC triggers for ARC PCLC 3275 may include low blood pressure. aTKT triggers for tourniquet aTKT PCLC 3280 may include placement and extremity blood. OxyClover triggers for OxyClover PCLC 3205 may include intubation and sedation. MechClover triggers for MechClover PCLC 3215 may include intubation and sedation. ACL triggers for anesthesia PCLC 3225 may include sedation.

Outputs for the various PCLCs may include as follows. OxyClover 3205 outputs may include FiO2; and PEEP. MechClover 3215 outputs may include RR-mechanical, RR-spontaneous, and tidal volume. Anesthesia 3225 ACL outputs may include anesthetic flow rat and anesthetic depth; aTKT 3280 outputs may include extremity bleed status; ARC 3275 outputs may include fluid flow rate and fluid responsiveness; and V-ARC 3270 outputs may include vasopressor flow rate and “stability” measurement.

SACM 3240 is moreover operable to provide one or more SACM alerts, which may for example include one or more of: active hemorrhage and resuscitation, alert to status every 500 mL; sustained low blood pressure (BP); at controller limits; v-ARC triggered; signal quality issues; high ventilator and low BP, alert to PTX; anesthesia rate very low; and total anesthesia greater than limit.

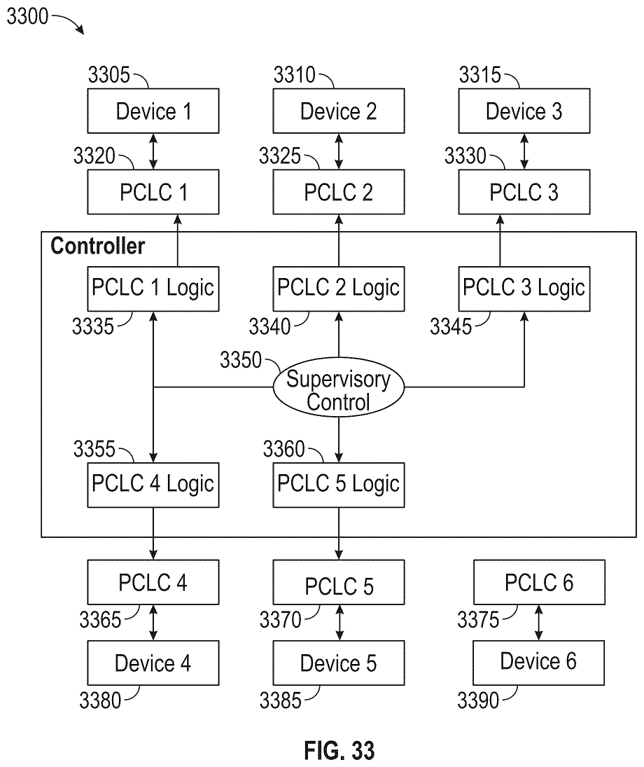

As shown in the system block diagram 3300 of FIG. 33, supervisory control 3350, such as that provided by a SACM, is used to control a number of PCLCs PCLC1 13320, PCLC2 3325, PCLC3 3330, PCLC4 3365, PCLC5 3370, PCLC6 3375 via corresponding PCLC logic blocks 3335, 3340, 3345, 3355, 3360; as shown in FIG. 32, PCLC logic blocks may be used to control one or more PCLCs as directed by the supervisory control 3350. The supervisory control 3350 and PCLC logic blocks 3335, 3340, 3345, 3355, 3360 may be part of a hardware controller that may be separated from or coupled to the respective controllers PCLCs PCLC1 13320, PCLC2 3325, PCLC3 3330, PCLC4 3365, PCLC5 3370, PCLC6 3375. The hardware controller may be a controller of a computer operable to execute instructions that reside on or off the associated hardware components. The PCLCs PCLC1 13320, PCLC2 3325, PCLC3 3330, PCLC4 3365, PCLC5 3370, PCLC6 3375 control corresponding autonomous devices for automated patient care, shown here as Device1 3305, Device2 3310, Device3 3315, Device4 3380, Device5 3385, and Device6 3390.

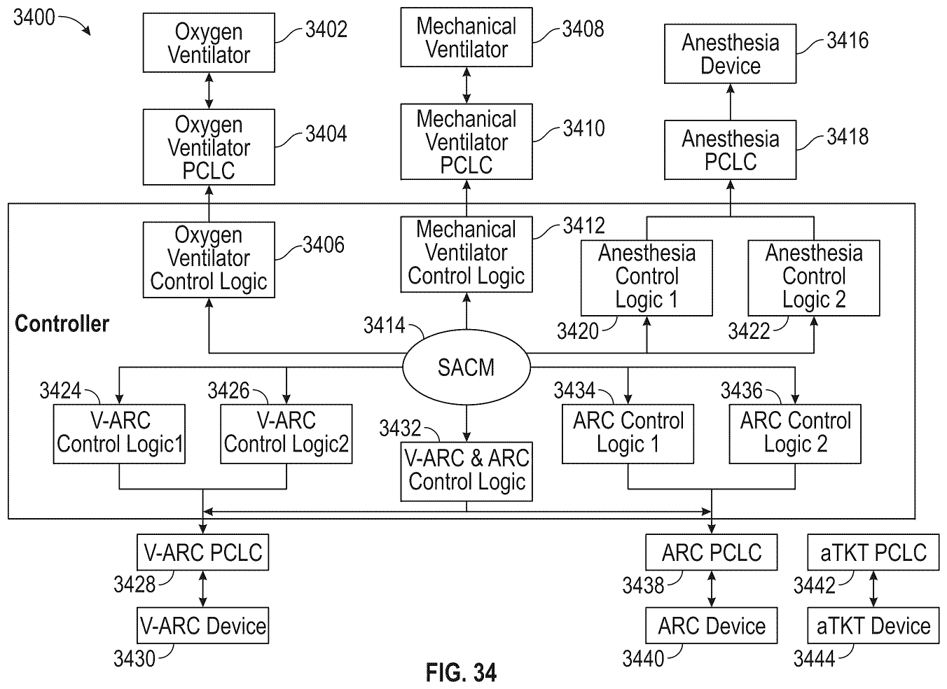

More particularly, the system 3400 of FIG. 34 includes oxygen ventilator 3402 controlled by oxygen ventilator PCLC 3404; mechanical ventilator 3408 controlled by mechanical ventilator PCLC 3410; anesthesia device 3416 controlled by anesthesia PCLC 3418; V-ARC device 3430 controlled by V-ARC PCLC 3428; ARC device 3440 controlled by ARC PCLC 3438; and aTKT device 3444 is controlled by aTKT PCLC 3442. Each of the PCLC has corresponding control logic that defines how control of the PCLC by the SACM is carried out. Thus, SACM 3414 carries out control of oxygen ventilator PCLC 3404 via oxygen ventilator control logic 3406; SACM 3414 controls mechanical ventilator PCLC 3410 via mechanical control logic 3412; SACM 3414 carries out control of anesthesia PCLC 3418 via one or more of anesthesia control logic1 3420 and anesthesia control logic2 3422; SACM 3414 controls V-ARC PCLC 3428 via one or more of V-ARC control logic1 3424, V-ARC control logic2 3426, and V-ARC & ARC control logic 3432; and SACM 3414 controls ARC PCLC 3438 via one or more of ARC control logic1 3424, V-ARC control logic2 3436, and V-ARC & ARC control logic 3432.

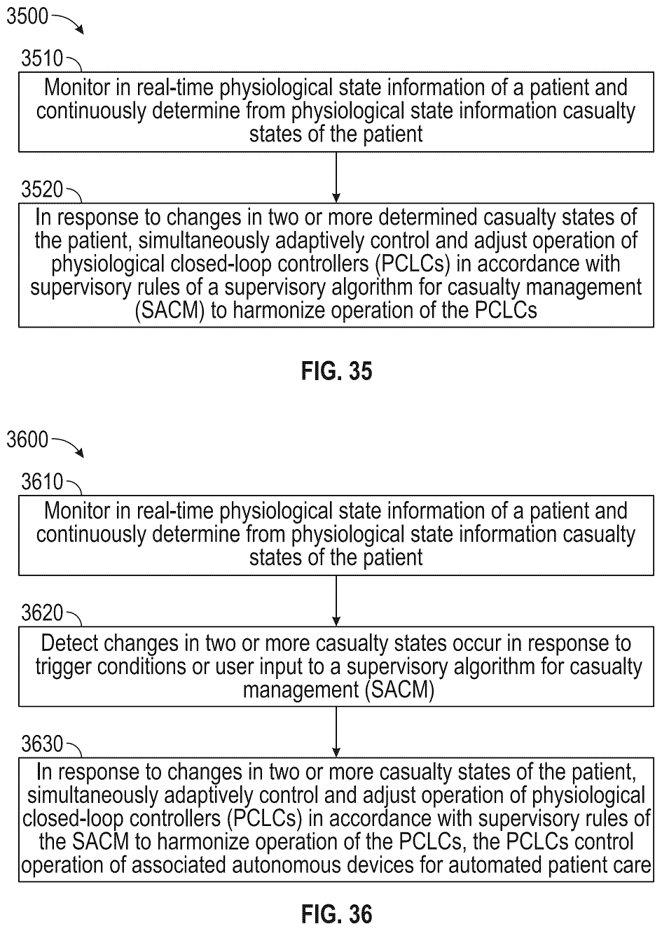

With regard to the methodologies described herein, reference is made to FIGS. 35 and 36. Referring now to flow 3500 of FIG. 35, physiological state information of a patient is monitored in real-time so that the casualty state(s) of the patient can be continuously determined and updated at 3510. At 3520, in response to changes in two or more casualty states of the patient, the simultaneously adaptive control and adjust operation of PCLCs in accordance with supervisory rules of a supervisory algorithm for casualty management (SACM) to harmonize operation of the PCLCs.

In flow 3600 of FIG. 36, at 3610 the physiological state information of a patient is monitored in real-time so that the casualty state(s) of the patient can be continuously determined and updated. Changes in two or more casualty states that occur in response to trigger conditions or user input to the SACM are detected at 3620. At 3630, in response to the detected changes at 3620, simultaneous adaptive control and adjust operation of PCLCs are performed, in accordance with supervisory rules of the SACM to harmonize operations of the PCLCs. As previously noted, the PCLCs control operation of associated autonomous devices for automated patient care.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a data processing system, or a computer program product.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system for adaptive supervisory control of physiologic closed-loop controllers, comprising:
a controller in communication with and operable to simultaneously control a plurality of physiological closed-loop controllers (PCLCs) of a corresponding plurality of autonomous devices for automated patient care and to receive in real-time a plurality of physiological state information of a patient detected by the plurality of autonomous devices; and a supervisory algorithm for casualty management (SACM) of the controller that monitors the physiological state information of the patient received by the controller, continuously determines a plurality of casualty states of the patient from the received physiological state information of the patient, and adaptively controls operation of the plurality of physiological closed-loop controllers (PCLCs) responsive to the determined casualty states of the patient, the SACM operable to simultaneously control and adjust operation of the PCLCs responsive to changes in two or more casualty states of the patient to harmonize operation of the PCLCs in accordance with a plurality of supervisory rules of the SACM.

2. The system of claim 1, where responsive to determining that two or more casualty state changes of the plurality of current casualty states of the patient trigger one or more supervisory rules, the SACM controls one or more of the plurality of PCLCs to harmonize operation of the PCLCs as a function of the two or more casualty state changes.

3. The system of claim 2, where the change in the two or more casualty states is triggered by one or more trigger conditions or a user input to the SACM of the controller and where the one or more trigger conditions are trigger conditions detected by one or more of the plurality of autonomous devices for autonomous patient care controlled by the plurality of PCLCs.

4. The system of claim 2, where the one or more supervisory rules are determined from one or more of clinical practice guidelines, safety limitations of one or more PCLCs of the plurality of PCLCs, and expertise of one or more subject matter experts (SMEs).

5. The system of claim 2, where the one or more supervisory rules include a set of logical rules of a rules engine of the SACM and where the rules engine is operable to receive one or more of setpoint values and parameters from the plurality of PCLCs and the physiological state information of the patient and to adjust settings of the rules engine responsively.

6. The system of claim 5, where the rules engine includes two or more logic rule categories including stable, shock, non-responsive, pneumothorax, hemorrhage, septic shock, traumatic brain injury, or prolonged care; criteria to enter a casualty state of the plurality of casualty states for each logic rule category; and control information used by the SACM to control operation of the plurality of PCLCs in accordance with the current plurality of casualty states of the patient.

7. The system of claim 1, where the plurality of casualty states include stable and one or more non-stable casualty states, the non-stable casualty states including one or more of hemorrhagic shock, traumatic brain injury, prolonged field care, septic shock, internal hemorrhage, external hemorrhage, pre-pneumothorax remedy, and pre-pneumothorax remedy.

8. The system of claim 7, where the hemorrhagic shock, septic shock and traumatic brain injury casualty states each further include two or more degree states indicating degree of severity.

9. The system of claim 1, where the changes in two or more casualty states of the patient include two or more of a change from the stable casualty state to a non-stable casualty state, a change from first to second non-stable casualty states, a change from one or more non-stable casualty states to the stable casualty state.

10. The system of claim 9, where the change in the two or more casualty states is triggered by one or more trigger conditions or a user input to the SACM of the controller and where the one or more trigger conditions are trigger conditions detected by one or more of the plurality of autonomous devices for autonomous patient care controlled by the plurality of PCLCs.

11. The system of claim 1, further comprising the plurality of PCLCs in cooperative communication with the SACM of the controller and each coupled to and operable to control a corresponding autonomous device of the plurality of autonomous devices, with each PCLC of the plurality of PCLCs having control logic circuitry operable to control the corresponding autonomous device for automated patient care, the respective autonomous device controlling one or more of fluid administration, vasopressor therapy, mechanical ventilation, oxygenation management, hemorrhage control, and anesthesia management of the patient.

12. The system of claim 11, further comprising the plurality of autonomous devices for automated patient care each controlled by a corresponding PCLC of the plurality of PCLCs to control an underlying physiological state of the patient.

13. The system of claim 12, each autonomous device operable to detect one or more of incoming vital readings, lab values and user input related to the underlying physiological state of the patient and provide said detected vital readings, lab values or user input to one or more of the corresponding PCLC and the SACM of the controller.

14. A method configured to provide supervisory control of physiologic closed-loop controllers, comprising:
monitoring in real-time physiological state information of a patient and continuously determining from the physiological state information a plurality of casualty states of the patient; and
in response to changes in two or more determined casualty states of the patient, simultaneously adaptively controlling and adjusting operation of a plurality of physiological closed-loop controllers (PCLCs) in accordance with a plurality of supervisory rules of a supervisory algorithm for casualty management (SACM) to harmonize operation of the PCLCs.

15. The method of claim 14, where the physiological state information of the patient is one or more vital readings, lab values and user input related to the underlying physiological state of the patient.

16. The method of claim 15, further comprising detecting one or more of the incoming vital readings, lab values and user input related to the underlying physiological state of the patient by one or more of a plurality of autonomous devices for automated patient care, the plurality of autonomous devices configured for controlling one or more of fluid administration, vasopressor therapy, mechanical ventilation, oxygenation management, hemorrhage control, and anesthesia management of the patient.

17. The method of claim 14, further comprising in response to changes in two or more determined casualty states of the patient the plurality of PCLCs adaptively controlling and adjusting operation of a corresponding plurality of autonomous devices for automated patient care that are controlled by the plurality of PCLCs, the plurality of autonomous devices configured for controlling one or more of fluid administration, vasopressor therapy, mechanical ventilation, oxygenation management, hemorrhage control, and anesthesia management of the patient.

18. The method of claim 14, further comprising triggering the changes in two or more determined casualty states in response to one or more trigger conditions or a user input to the SACM, the SACM coupled to and controlled by a controller that controls operations of the plurality of PCLCs.

19. The method of claim 18, further comprising detecting the one or more trigger conditions by one or more of a plurality of autonomous devices for autonomous patient care controlled by the plurality of PCLCs, the plurality of autonomous devices configured for controlling one or more of fluid administration, vasopressor therapy, mechanical ventilation, oxygenation management, hemorrhage control, and anesthesia management of the patient.

20. The method of claim 14, further comprising determining the one or more supervisory rules from one or more of clinical practice guidelines, safety limitations of one or more PCLCs of the plurality of PCLCs, and expertise of one or more subject matter experts (SMEs).

21. The method of claim 14, where the one or more supervisory rules include a set of logical rules of a rules engine of the SACM and further including the rules engine receiving one or more of setpoint values and parameters from the plurality of PCLCs and the physiological state information of the patient and adjusting settings of the rules engine responsively.

22. The method of claim 14, where the changes in two or more casualty states of the patient include two or more of a change from the stable casualty state to a non-stable casualty state, a change from first to second non-stable casualty states, a change from one or more non-stable casualty states to the stable casualty state, the method including changing the two or more casualty state in response to one or more trigger conditions or a user input to the SACM.

23. The method of claim 22, further including detecting the one or more trigger conditions by one or more of a plurality of autonomous devices for autonomous patient care controlled by the plurality of PCLCs, the plurality of autonomous devices configured for controlling one or more of fluid administration, vasopressor therapy, mechanical ventilation, oxygenation management, hemorrhage control, and anesthesia management of the patient.
